(19) United States
(12) Patent Application Publication
(10) Pub. No.: US 2021/0238173 A1
(43) Pub. Date: Aug. 5, 2021

(54) MITOGEN-ACTIVATED PROTEIN KINASE KINASE 7 INHIBITORS

(71) Applicant: Yeda Research and Development Co. Ltd., Rehovot (IL)

(72) Inventors: Nir LONDON, Rehovot (IL); Amit SHRAGA, Rehovot (IL); Evgenia OLSHVANG, Rehovot (IL)

(73) Assignee: Yeda Research and Development Co. Ltd., Rehovot (IL)

(21) Appl. No.: 16/972,013

(22) PCT Filed: Jun. 4, 2019
(86) PCT No.: PCT/IL2019/050638

§ 371 Date: Dec. 4, 2020

(30) Foreign Application Priority Data
Jun. 4, 2018 (IL) 259810

(51) Int. Cl. C07D 471/04; C07D 231/56; C07D 491/052; C07D 401/04

(52) U.S. Cl. CPC C07D 471/04; C07D 401/04; C07D 491/052; C07D 231/56

(57) ABSTRACT

Provided are compounds that act as covalent inhibitors of mitogen-activated protein kinase kinase 7 (MKK7 enzyme), method of preparation and uses thereof.

### RELATED APPLICATION

This application claims the benefit of priority of Israeli Patent Application No. 259810 filed 4 Jun. 2018, the contents of which are incorporated herein by reference in their entirety.

### FIELD AND BACKGROUND OF THE INVENTION

The present invention, in some embodiments thereof, relates to pharmaceutical chemistry and, more particularly, but not exclusively, to a family of MKK7 inhibitors and uses thereof.

Covalent targeting of non-conserved cysteine residues in kinase active sites has proven a robust strategy to achieve both potency and more importantly selectivity across the kinome. Several approved drugs resulted from such efforts, including the covalent kinase inhibitors: ibrutinib, afatinib, osimertinib and neratinib.

MKK7, also known as MAP2K7, is one of two upstream activators of JNKs. Like other mitogen activated protein kinase (MAPK) cascades, the JNK cascade is composed of three tiers. Following an extra cellular stress signal such as UV, osmotic shock, exposure to cytokines, or toxins, a variety of receptors such as TLR4, IL-1 receptor and TNFα receptor will activate one or several MAPK kinase kinases (MAP3Ks). Canonical MAP3Ks for the JNK pathway are for example DLK, MLK, TAK and ASK1. These MAP3Ks will in turn phosphorylate and activate one or both MAPK kinases (MAP2Ks) MKK7 and MKK4. To fully activate JNK, MKK7 and MKK4 will phosphorylate Thr-183 and Tyr-185 on JNK's activation loop. This entire cascade, including sometimes the substrates of JNK such as cJUN, ATF2, and p53 are co-localized by binding to a scaffold protein: JIP (JNK interacting Protein).

Since the JNK pathway is so central to numerous cellular processes and particularly inflammatory processes, it is considered a therapeutic target for a variety of indications. For instance, rheumatoid arthritis, inflammatory bowel disease and Alzheimer's disease. Previous studies opted to directly inhibit JNK, either via inhibitors such as the promiscuous SP600125, or more recently, with selective inhibitors. However, there is no approved drug targeting JNK, likely due to its wide expression profile in all tissues. Conversely, MAP3K were considered as candidate targets, as inhibiting upstream would potentially inhibit the activation of the entire JNK pathway. Still, inhibiting MAP3K might not be effective due to potential redundancy.

A recent study [Chang, C.-F. et al., European Journal of Medicinal Chemistry, 2016, 124, pp. 186-199; referred to hereinafter as “the Chang study” ], has focused on Aurora kinase, which have emerged as anticancer targets, and on inhibitors thereof, a few of which have advanced into clinical study. The study utilized in silico fragment-based approach and knowledge-based drug design to identify compounds that act as specific Aurora A and/or B inhibitors, based on interaction with specific residues in the Aurora kinase binding pocket, particularly targeting residues Arg220, Thr217 or Glu177.

Additional background art includes U.S. Pat. Nos. 9,227,978, 8,088,783, 7,514,444, 7,195,894, 7,803,749, and 6,136,596, and U.S. Patent Application Publication Nos. 2010/0004234, 2006/0079494, 2005/0009876 and 2004/0243224, WO/2014/130693, WO/2016/004272, WO/2016/133935, WO/2016/010108, JP2011/246389, Lanning, B. R. et al., Nat. Chem. Biol., 2014, 10(9), pp. 760-767 and Zhao, Z. et al., J. Med. Chem., 2017, 60, pp. 2879-2889.

### SUMMARY OF THE INVENTION

Aspect so the present invention are drawn to covalent inhibitors of MKK7. The c-Jun NH₂-terminal kinase (JNK) signaling pathway is central to the cell response to stress, inflammatory signals and toxins. While selective inhibitors are known for JNKs themselves and for various MAP3Ks in the pathway, no selective inhibitor is known for MKK7 hitherto—one of the two direct MAP2Ks that activate JNK. Based on covalent virtual screening, the present inventors have identified the first reported selective MKK7 covalent inhibitors, presented herein. The inhibitors presented herein were optimized to low-micromolar cellular inhibition of JNK phosphorylation, based on a design paradigm that is focused on a non-conserved cysteine residue in the active site of MKK7, which is missing in other kinases, such as Aurora kinases. The crystal structure of an exemplary inhibitor, according to embodiments of the present invention, has been determined, and corroborated that the covalent virtual screening correctly predicted the mode of binding. The molecular optimization asserted the selectivity of the inhibitors presented herein on a proteomic level and against a panel of 76 kinases, and validated on-target effect using knockout cell lines. Additionally, the inhibitors presented herein were shown to block activation of primary mouse B-cells in response to lipopolysaccharide. The covalent inhibitor compounds presented herein allow the investigation of JNK signaling and can serve as leads for the development of therapeutic drugs.

According to an aspect of some embodiments of the present invention there is provided a compound of general formula I′:

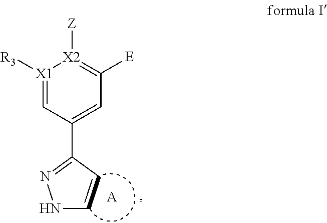

and any enantiomer, solvate, hydrate or a pharmaceutical acceptable salt thereof, wherein:

E is a reactive electrophile moiety

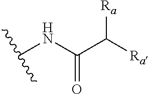

wherein each of Rₐ and R_{a′} is independently selected from the group consisting of H, F, Cl, Br, Me, Et and Pr, or a reactive electrophile moiety selected from the group consisting of:

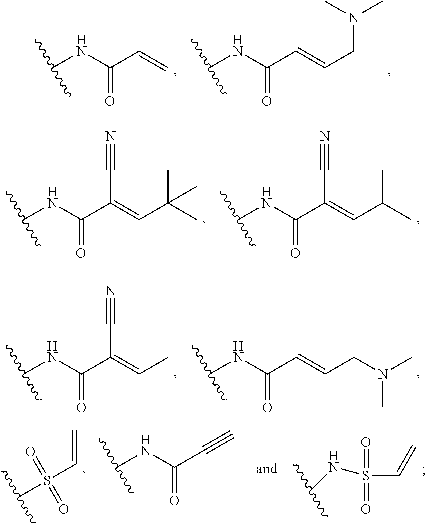

R₃ is H or a substituent selected from the group consisting of:

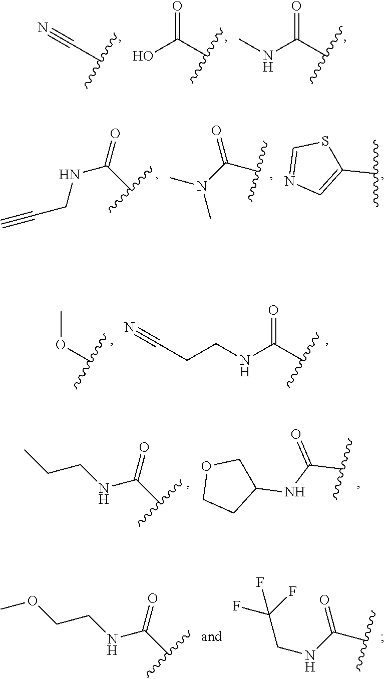

X₁ and X₂ are each independently C and N;

Z is C or H; and

ring A is a 5- or 6-membered, aromatic or aliphatic, substituted or unsubstituted ring having 0-2 heteroatoms therein, selected from the group consisting of:

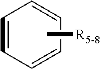

wherein each of R₅₋₈ is independently selected from the group consisting of H, F, Cl, Br, NO₂, Me, OMe and Ph, or ring A is selected from the group consisting of:

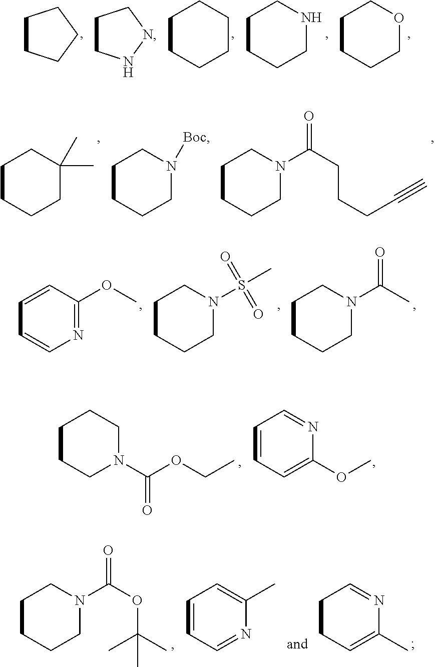

with the proviso that the compound is not selected from the group consisting of:
(Z)-4-oxo-4-((3-(6-(phenylsulfonamido)-1H-indazol-3-yl)phenyl)amino)but-2-enoicacid,
(Z)-4-oxo-4-((3-(6-(p-tolyl)-1H-indazol-3-yl)phenyl)amino)but-2-enoicacid,
N-methyl-N-[2-oxo-2-[[3-(4,5,6,7-tetrahydro-1H-indazol-3-yl)phenyl]amino]ethyl]-2-propenamide,
N-[3-oxo-3-[[3-(4,5,6,7-tetrahydro-1H-indazol-3-yl)phenyl]amino]propyl]-2-propenamide,
3-[5-(4,5,6,7-tetrahydro-5-methyl-1H-pyrazolo[4,3-c]pyridin-3-yl)-3-pyridinyl]-2-propenoicacid ethyl ester,

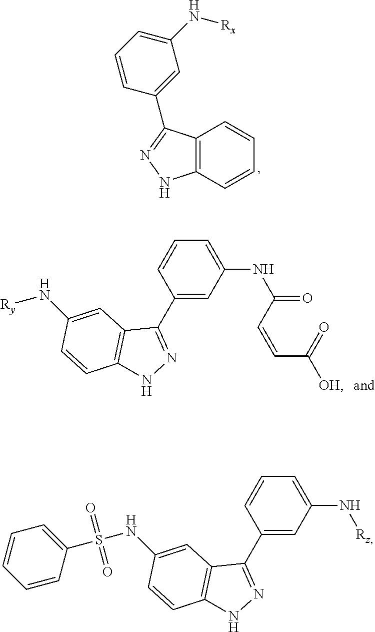

wherein:
Rₓ is selected from the group consisting of

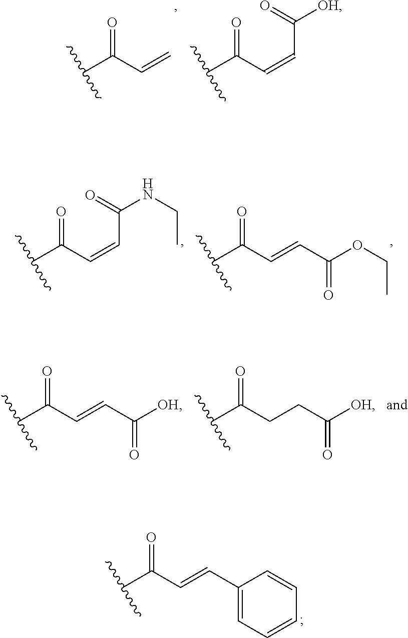

R_{y} is selected from the group consisting of PhNHCO, 4-Ph-PhSO₂, PhCO, 4-tBu-PhSO₂, PhCH₂, 4-OCH₃-PhSO₂, PhSO₂, 4-NO₂-PhSO₂, 4-CH₃-PhSO₂, and 4-F-PhSO₂; and

R_{z} is selected from the group consisting of

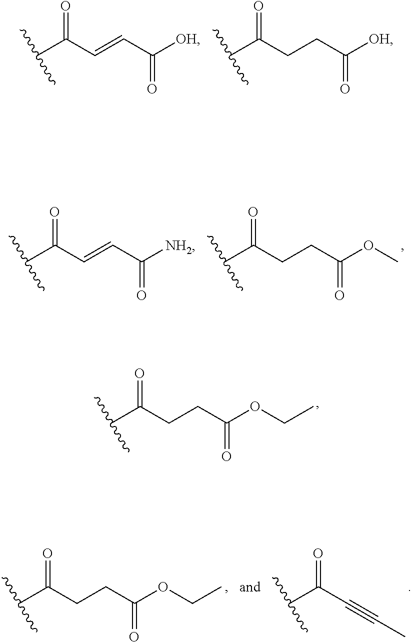

According to another aspect of some embodiments of the present invention there is provided a compound represented by general formula I:

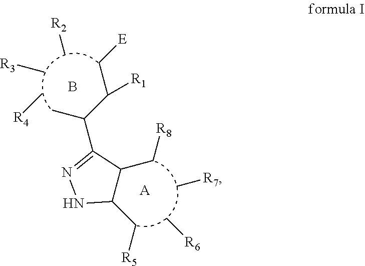

and any enantiomer, solvate, hydrate or a pharmaceutical acceptable salt thereof, wherein:

each of ring A and ring B is independently a 5- or 6-membered aromatic or aliphatic ring having 0-2 heteroatoms therein;

E is a reactive electrophile moiety; and

each of R₁-R₈ is independently absent, H or a substituent,

with the proviso that the compound is not selected from the group consisting of:
(Z)-4-oxo-4-((3-(6-(phenylsulfonamido)-1H-indazol-3-yl)phenyl)amino)but-2-enoic acid,
(Z)-4-oxo-4-((3-(6-(p-tolyl)-1H-indazol-3-yl)phenyl)amino)but-2-enoic acid,
N-methyl-N-[2-oxo-2-[[3-(4,5,6,7-tetrahydro-1H-indazol-3-yl)phenyl]amino]ethyl]-2-propenamide,
N-[3-oxo-3-[[3-(4,5,6,7-tetrahydro-1H-indazol-3-yl)phenyl]amino]propyl]-2-propenamide,
3-[5-(4,5,6,7-tetrahydro-5-methyl-1H-pyrazolo[4,3-c]pyridin-3-yl)-3-pyridinyl]-2-propenoic acid ethyl ester,

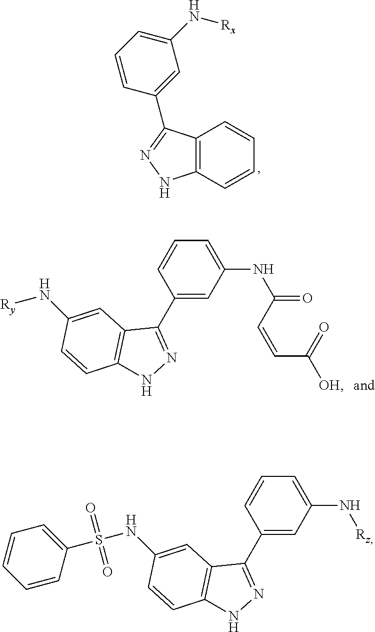

wherein:

Rₓ is selected from the group consisting of

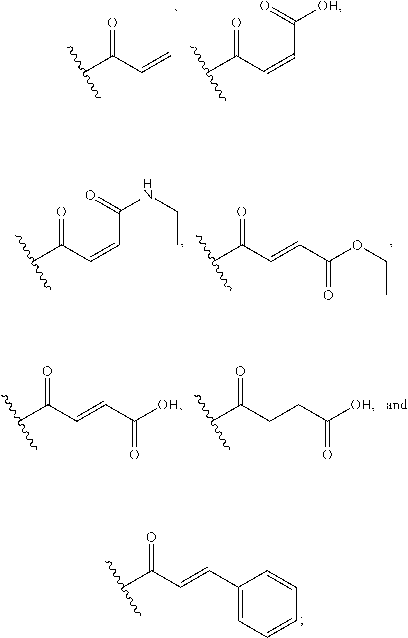

R_{y} is selected from the group consisting of PhNHCO, 4-Ph-PhSO₂, PhCO, 4-tBu-PhSO₂, PhCH₂, 4-OCH₃-PhSO₂, PhSO₂, 4-NO₂-PhSO₂, 4-CH₃-PhSO₂, and 4-F-PhSO₂; and

R_{z} is selected from the group consisting of

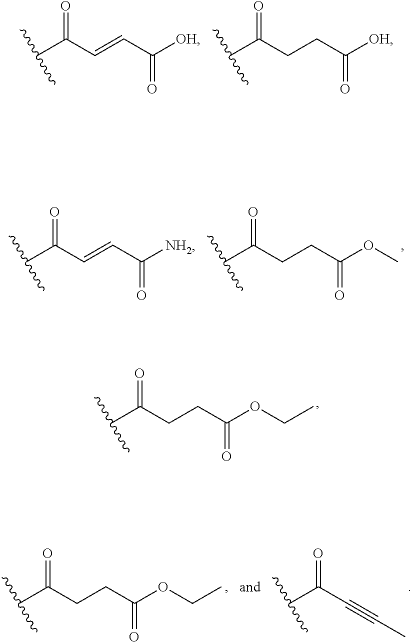

According to some embodiments of the invention, the reactive electrophile moiety is capable of forming a covalent bond with a side-chain of residue that corresponds to a cysteine at position 218 of an MKK7 enzyme.

According to some embodiments of the invention, the compound provided herein is capable of inhibiting human MKK7 enzyme by bonding covalently to CYS218 thereof.

According to some embodiments of the invention, the compound provided herein is characterized by exhibiting inhibition of human MKK7 enzyme at a concentration lower by at least two orders of magnitude compared to an Aurora kinase enzyme.

According to some embodiments of the invention, the reactive electrophile moiety, E in formula I, can be represented by general formula II:

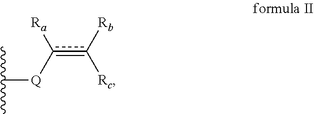

wherein:

Q is selected from the group consisting of —C(═O)—, —OC(═O)—, —CH₂C(═O)—, —NR_{d}C(═O)—, —P(OR_{d})(═O)—, —CH₂NR_{d}C(═O)—, —S(═O)—, —CH₂S(═O)₂—, —S(═O)₂—, and —NR_{d}S(═O)₂—;

R_{d} is H, C₁₋₆ alkyl or hydroxylalkyl;

[symbol] is a carbon-carbon double bond or a carbon-carbon triple bond;

each of Rₐ, R_{b} and R_{c} is independently absent, H, or selected from the group consisting of C₁₋₆ alkyl, aminoalkyl, alkylaminoalkyl, cyano, and hydroxylalkyl, or Rₐ and R_{b} join to form a alicyclic or heterocyclic ring when [symbol] is a is a double bond; or Rₐ is absent and R_{b} and/or R_{c} is H, C₁₋₆ alkyl, aminoalkyl, alkylaminoalkyl or hydroxylalkyl when [symbol] is a triple bond.

According to some embodiments of the invention, the reactive electrophile moiety is selected from the group consisting of:

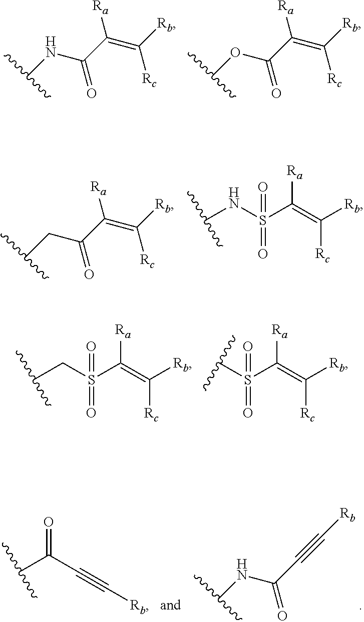

According to some embodiments of the invention, Rₐ is an electron withdrawing group.

According to some embodiments of the invention, Q is —NHC(═O)—.

According to some embodiments of the invention, each of Rₐ, R_{b} and R_{c} is H.

According to some embodiments of the invention, the reactive electrophile moiety is selected from the group consisting of:

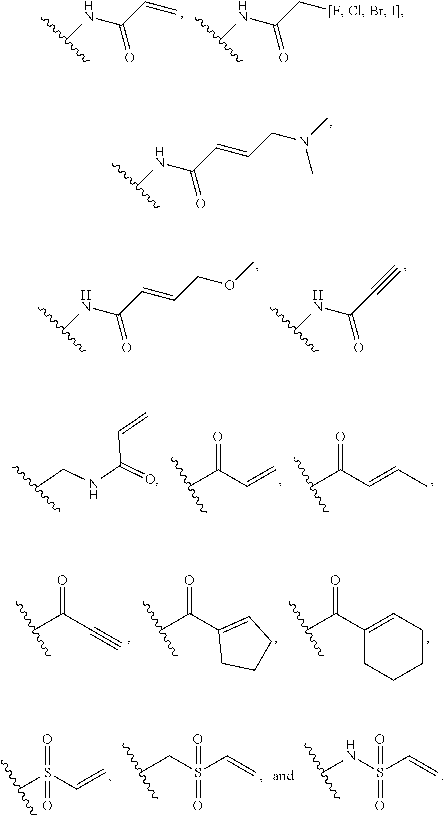

According to some embodiments of the invention, ring A is a 6-membered alicyclic, heteroalicyclic, aryl or heteroaryl.

According to some embodiments of the invention, ring B is a 6-membered alicyclic, heteroalicyclic, aryl or heteroaryl.

According to some embodiments of the invention, ring A and ring B are each a 6-membered aryl.

According to some embodiments of the invention, the substituent(s) on ring A and/or ring B is individually selected from the group consisting of a halo, a cyano, a nitrile, a nitro, a C₁₋₆ alkyl, a C₁₋₆ alkenyl, an alkynyl, germinal C₁₋₆ alkanes, a benzyl, an aryl, a heteroaryl, an alkoxy, an aryloxy, a carbonyl, a carboxyl, a carboxylate, an amide, an alkylsulfonyl, a heterobicycle, a bi-phenyl, a substituted bi-phenyl, a bi-aryl, and a substituted bi-aryl.

According to some embodiments of the invention, the compound provided herein is characterized by an octanol-water partition coefficient (Log P_{ow}) value that ranges from −1 to 6.

According to some embodiments of the invention, the compound provided herein is selected from the group consisting of:

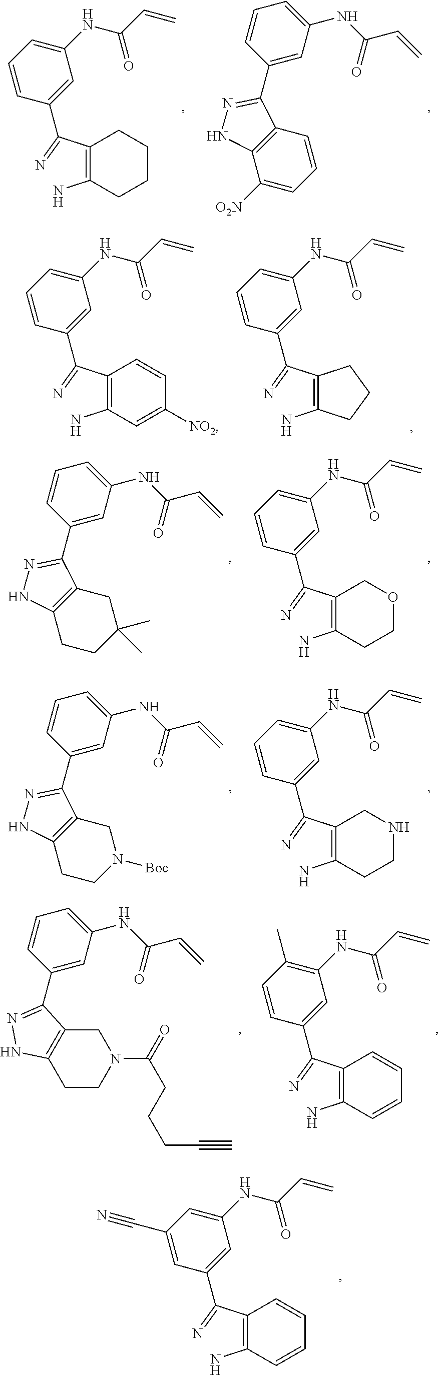

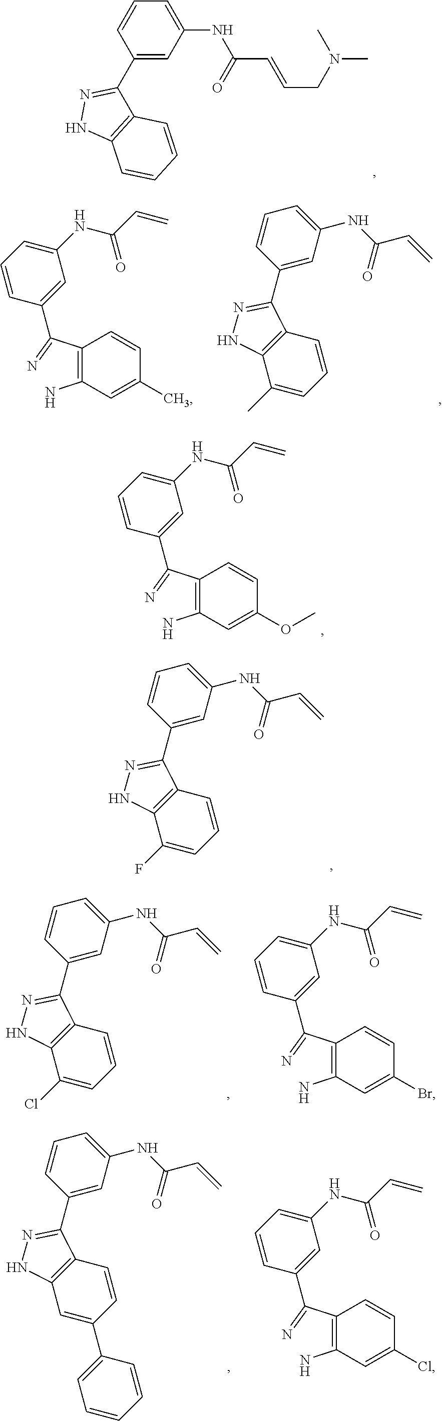

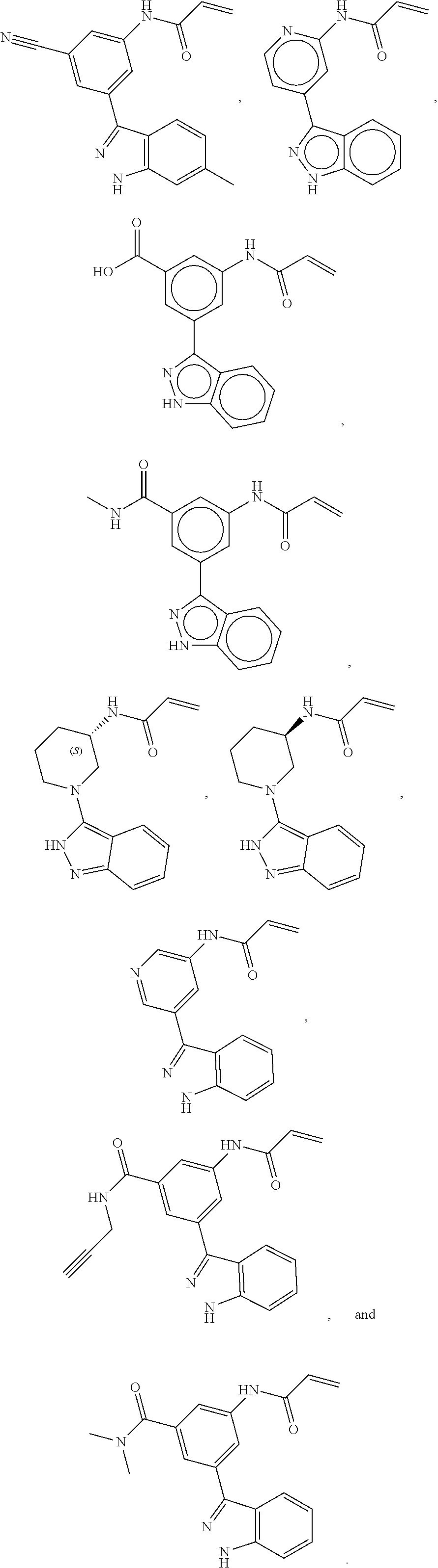

According to some embodiments of the invention, the compound provided herein is selected from the group consisting of:

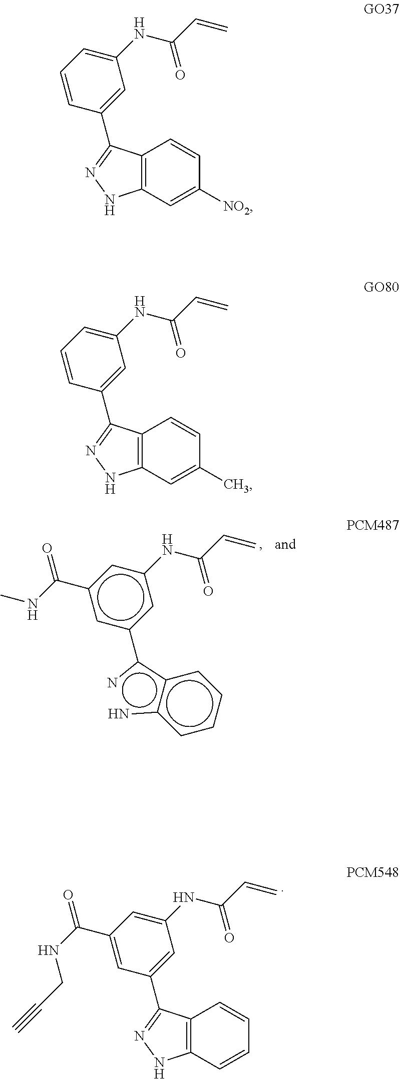

According to an aspect of some embodiments of the present invention there is provided a pharmaceutical composition that includes, as an active ingredient, a compound represented by general formula I:

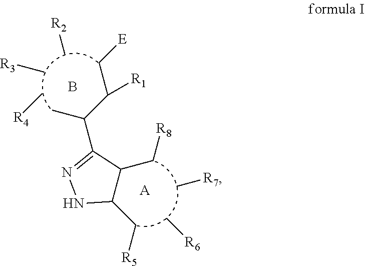

and any enantiomer, solvate, hydrate or a pharmaceutical acceptable salt thereof, wherein:

each of ring A and ring B is independently a 5- or 6-membered aromatic or aliphatic ring having 0-2 heteroatoms therein;

E is a reactive electrophile moiety; and

each of R₁-R₈ is independently absent, H or a substituent,

and a pharmaceutically acceptable carrier, packaged in a packaging material and identified in print for use in the treatment of a medical condition, a disease or a disorder associated with c-Jun-NH₂-terminal kinase (JNK) pathway regulation.

According to some embodiments of the invention, the medical condition, disease or disorder is associated with MKK7 enzyme. In some embodiments, the MKK7 enzyme is having a cysteine residue corresponding to Cys218 in human MKK7.

According to some embodiments of the invention, the medical condition, disease or disorder is associated not associated with an Aurora kinase.

According to an aspect of some embodiments of the present invention there is provided a method of treating a medical condition, disease or disorder associated with c-Jun-NH₂-terminal kinase (JNK) pathway regulation, that includes administering to a subject in need thereof a therapeutically effective amount of a compound having general formula I:

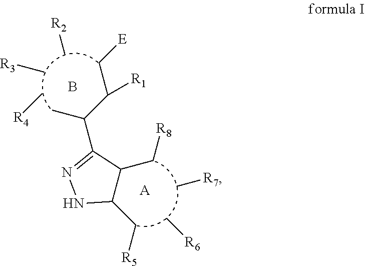

and any enantiomer, solvate, hydrate or a pharmaceutical acceptable salt thereof, wherein:

each of ring A and ring B is independently a 5- or 6-membered aromatic or aliphatic ring having 0-2 heteroatoms therein;

E is a reactive electrophile moiety; and

each of R₁-R₈ is independently absent, H or a substituent.

According to some embodiments of the invention, relating to the method of treatment, the medical condition, disease or disorder is associated with MKK7 enzyme, and more specifically to MKK7 enzyme that exhibits a cysteine residue at the position corresponding to Cys218 of human MKK7.

According to yet another aspect of some embodiments of the present invention, there is provided a use of a compound having general formula I:

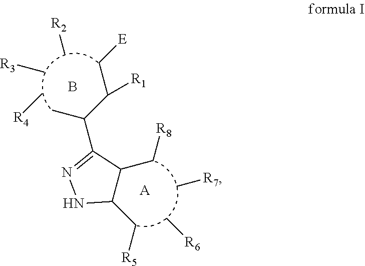

and any enantiomer, solvate, hydrate or a pharmaceutical acceptable salt thereof, wherein:

each of ring A and ring B is independently a 5- or 6-membered aromatic or aliphatic ring having 0-2 heteroatoms therein;

E is a reactive electrophile moiety; and

each of R₁-R₈ is independently absent, H or a substituent,

for the preparation of a medicament for the treatment of a medical condition, disease or disorder associated with c-Jun-NH₂-terminal kinase (JNK) pathway regulation.

According to some embodiments of the invention, relating to any use of the compounds presented herein, the medical condition, disease or disorder is associated with MKK7 enzyme, and more specifically to MKK7 enzyme that exhibits a cysteine residue at the position corresponding to Cys218 of human MKK7.

According to some embodiments of the invention, relating to a pharmaceutical composition, method of treatment or use of the compound represented by formula I, the reactive electrophile moiety E in formula I is capable of forming a covalent bond with a side-chain of residue that corresponds to a cysteine at position 218 of an MKK7 enzyme.

According to some embodiments of the invention, relating to a pharmaceutical composition, method of treatment or use of the compound represented by formula I is capable of inhibiting human MKK7 enzyme by bonding covalently to CYS218 thereof.

According to some embodiments of the invention, relating to a pharmaceutical composition, method of treatment or use of the compound represented by formula I is characterized by exhibiting inhibition of human MKK7 enzyme at a concentration lower by at least two orders of magnitude compared to an Aurora kinase enzyme.

According to some embodiments of the invention, relating to a pharmaceutical composition, method of treatment or use of the compound represented by formula I, the reactive electrophile moiety E in formula I is represented by general formula II:

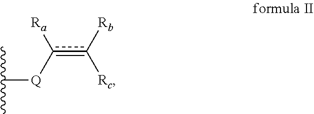

wherein:

Q is selected from the group consisting of —C(═O)—, —OC(═O)—, —CH₂C(═O)—, —NR_{d}C(═O)—, —P(OR_{d})(═O)—, —CH₂NR_{d}C(═O)—, —S(═O)—, —CH₂S(═O)₂—, —S(═O)₂—, and —NR_{d}S(═O)₂—;

R_{d} is H, C₁₋₆ alkyl or hydroxylalkyl;

[symbol] is a carbon-carbon double bond or a carbon-carbon triple bond;

each of Rₐ, R_{b} and R_{c} is independently absent, H, or selected from the group consisting of C₁₋₆ alkyl, aminoalkyl, alkylaminoalkyl, cyano, and hydroxylalkyl, or Rₐ and R_{b} join to form a alicyclic or heterocyclic ring when [symbol] is a is a double bond; or Rₐ is absent and R_{b} and/or R_{c} is H, C₁₋₆ alkyl, aminoalkyl, alkylaminoalkyl or hydroxylalkyl when [symbol] is a triple bond.

According to some embodiments of the invention, relating to a pharmaceutical composition, method of treatment or use of the compound represented by formula I, the medical condition, disease or disorder is selected from the group consisting of Parkinson's disease, Alzheimer's disease, Pick's disease, Crohn's disease, Behcet's disease, stroke, coronary artery disease, heart failure, abdominal aortic aneurysm, noonan syndrome, chronic hepatitis C virus infection, acute liver injury, non-alcoholic fatty liver disease, asthma, chronic obstructive pulmonary disease, amyotrophic lateral sclerosis, inflammatory bowel disease, polyglutamine disease, auditory hair cell degeneration, rheumatoid arthritis, systemic lupus eryththematosus, celiac disease, colorectal cancer, retinoblastoma, melanoma, breast carcinoma, ovarian cancer, obesity, insulin resistant, progressive supranuclear palsy, corticobasal degeneration, argyrophilic grain disease, familial frontotemporal dementia, and type 2 diabetes.

According to some embodiments of the invention, relating to a pharmaceutical composition, method of treatment or use of the compound represented by formula I, the medical condition, disease or disorder associated with MKK7 enzyme, with the proviso that the medical condition, disease or disorder is not diabetes, cancer, or inflammation.

According to some embodiments of the invention, relating to a pharmaceutical composition, method of treatment or use of the compound represented by formula I, the medical condition, disease or disorder associated with MKK7 enzyme, with the proviso that the medical condition, disease or disorder is not glioma, breast, ovarian, colon, and thyroid cancers.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### DESCRIPTION OF SOME SPECIFIC EMBODIMENTS OF THE INVENTION

The principles and operation of the present invention may be better understood with reference to the descriptions and accompanying schemes.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

As discussed hereinabove, there are only few known kinase inhibitors that inhibit MKK7, yet there is no reported potent and selective inhibitor of MKK7 hitherto. Since MKK7 is a pivotal enzyme in the mechanism of several medical conditions, there is a benefit in providing selective MKK7 inhibitors. In an attempt to design a selective MKK7 inhibitor, the present inventors have searched for a covalent inhibitor that would be effective in the nanomolar range. While searching such a family of selective and covalent MKK7 inhibitors, the present inventors have used structure-based drug design tools, which lead to afford a family of compounds that has demonstrated to operate covalently on MKK7, and that exhibit high selectivity at both on the kinome and the proteome level. It was further demonstrated that members of this inhibitor family are able to block activation of primary B-cells, thus representing both chemical tools as well as potential leads for future therapeutic drugs.

The challenging before the present inventors has been to design a compound that would balance potent reversible recognition, with an optimal placement of a mild yet reactive electrophile, such as acrylamide. To address this challenge, the present inventors have utilized DOCKovalent, a covalent virtual screening software, which allows, given a structural model of a protein and a target nucleophile, to screen arbitrarily large virtual libraries of electrophiles to prioritize the design of potent selective binders.

A Covalent MKK7 Inhibitor

According to an aspect of embodiments of the present invention, there is provided a compound, and any enantiomer, solvate, hydrate or a pharmaceutical acceptable salt thereof, that can be represented by the general formula I presented hereinbelow, wherein the common structural features include a 1H-pyrazole moiety fused to ring A, and linked to ring B:

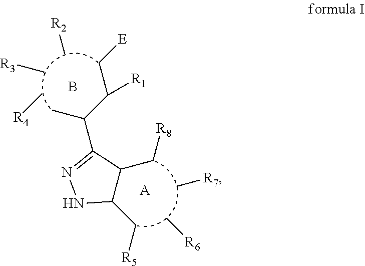

wherein each of ring A and ring B can be a 5- or 6-membered aromatic or alicyclic ring having 0-2 heteroatoms therein (heteroarylic or heteroalicyclic if heteroatoms are present in the ring). In general, each of R₁-R₈ can be, each independently, absent, H or a substituent, as defined and exemplified hereinbelow.

For example, according to some embodiments of the present invention, the compound may exhibit, without limitation, any one of the following structural skeleton:

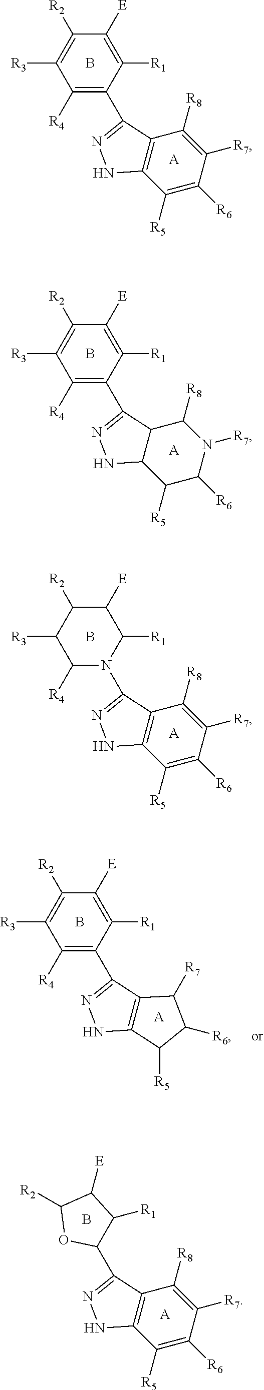

Another common structural feature of the compound is the presence of a reactive electrophile moiety, denoted E in formula I, at the specified position on ring B. “Electrophile” or “electrophilic moiety” is any moiety capable of reacting with a nucleophile (e.g., a moiety having a lone pair of electrons, a negative charge, a partial negative charge and/or an excess of electrons, for example a —SH group). Electrophiles typically are electron poor or comprise atoms which are electron poor. In certain embodiments an electrophile contains a positive charge or partial positive charge, has a resonance structure which contains a positive charge or partial positive charge or is a moiety in which delocalization or polarization of electrons results in one or more atom which contains a positive charge or partial positive charge. In some embodiments, the electrophiles comprise conjugated double bonds, for example an α,β-unsaturated carbonyl or α,β-unsaturated thiocarbonyl compound. In the context of embodiments of the present invention, the electrophile (E in formula I) is a functional group that in general is selected for its capability to form a covalent bond with another functional group, and more specifically, E is selected for its capability to form a covalent bond with the thiol of a cysteine residue of MKK7, namely the side-chain thiol of a cysteine residue that corresponds to the cysteine at position 218 of the human MKK7 protein. It is noted herein that the design target of the compounds presented herein is the human MKK7, and more specifically, the non-conserved cysteine at position 218 therein, thus the design paradigm underlying the present invention is the high-level selectivity and specificity of the presently disclosed compounds towards human MKK7; this feat is largely achieved as can be seen in Table 6 below, wherein it is clearly seen that exemplary members of the presently provided family of compounds serve as poor or null inhibitors of the kinase space referred to as the human kinome.

In some embodiments, the reactive electrophile moiety, E in formula I, has general formula H:

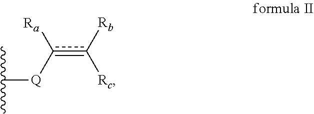

wherein:

Q is selected from the group consisting of —C(═O)—, —OC(═O)—, —CH₂C(═O)—, —NR_{d}C(═O)—, —P(OR_{d})(═O)—, —CH₂NR_{d}C(═O)—, —S(═O)—, —CH₂S(═O)₂—, —S(═O)₂—, and —NR_{d}S(═O)₂—;

R_{d} is H, C₁₋₆ alkyl or hydroxylalkyl;

[symbol] is a carbon-carbon double bond or a carbon-carbon triple bond;

each of Rₐ, R_{b} and R_{c} is independently absent, H, or selected from the group consisting of C₁₋₆ alkyl, aminoalkyl, alkylaminoalkyl, cyano, and hydroxylalkyl, or Rₐ and R_{b} join to form a alicyclic or heterocyclic ring when [symbol] is a is a double bond; or Rₐ is absent and R_{b} and/or R_{c} is H, C₁₋₆ alkyl, aminoalkyl, alkylaminoalkyl or hydroxylalkyl when [symbol] is a triple bond.

For example, the reactive electrophile moiety may be characterized by any one of the following structures:

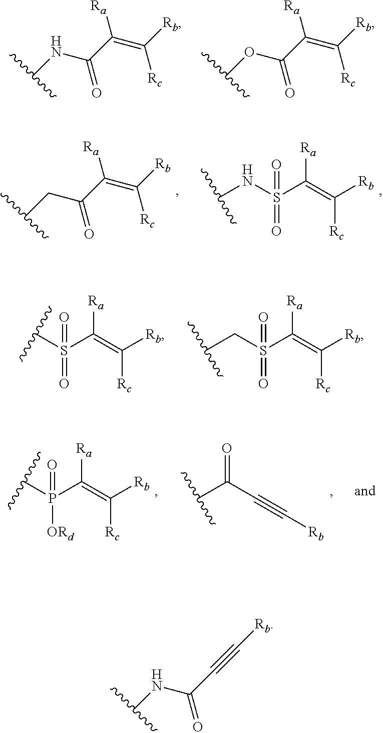

In some embodiments of the present invention, Rₐ exhibits an electron withdrawing group. According to embodiment soft the present invention, an electron withdrawing group (EWG) draws electrons away from a reaction center. When this center is an electron rich carbanion or an alkoxide anion, the presence of the electron-withdrawing substituent has a stabilizing effect. EWG include, without limitation and in a decreasing order of polar effect strength, moieties such as alkylsulfonyl (—SO₂R), triflyl (—SO₂CF₃), trihalo (—CF₃, —CCl₃), cyano (—C≡N), sulfonate (—SO₃H), nitro (—NO₂), ammonium (—NH₃⁺), quaternary amine (—NR₃⁺), aldehyde (—CHO), ketone (—COR), carboxyl (—COOH), acyl halide (—COCI, —COBr), ester (—COOR), amide (—CONH₂), and halo (—F, —Cl, —Br).

In some embodiments of the present invention, the reactive electrophile moiety, E, is an acrylamide, wherein Q is —NHC(═O)—, namely E is:

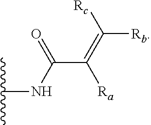

According to some embodiments of the present, when Q is —NHC(═O)—, each of Rₐ, R_{b} and R_{c} is H, and E is

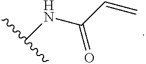 [SMILES: *NC(=O)C=C]

Alternatively, according to some embodiments, the reactive electrophile moiety, E, is selected from the group consisting of:

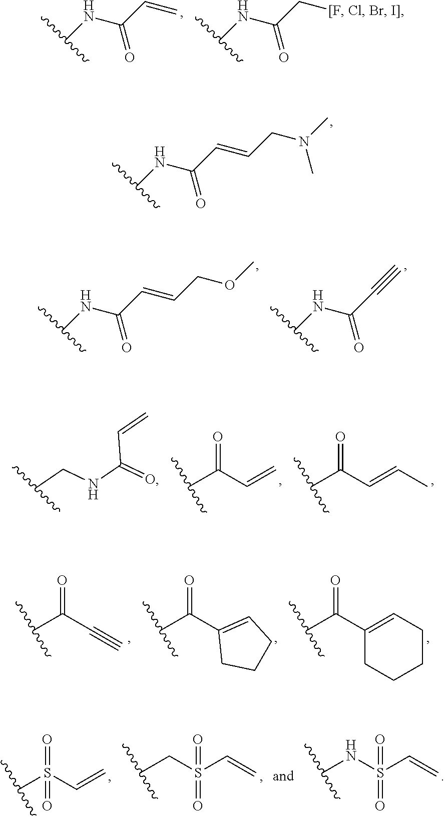

In some embodiments of the present invention, ring A is a 6-membered alicyclic, heteroalicyclic, aryl or heteroaryl. In some embodiments, ring A is a substituted or unsubstituted 6-membered aryl.

In some embodiments of the present invention, ring B is a 6-membered alicyclic, heteroalicyclic, aryl or heteroaryl. In some embodiments, ring B is a substituted or unsubstituted 6-membered aryl.

In some embodiments of the present invention, both ring A and ring B are each a substituted or unsubstituted 6-membered aryl.

In some embodiments, ring A is a substituted or unsubstituted 6-membered alicyclic, and ring B is a substituted or unsubstituted 6-membered aryl. In some embodiments, ring B is a substituted or unsubstituted 6-membered alicyclic, and ring A is a substituted or unsubstituted 6-membered aryl. In some embodiments, ring A is a substituted or unsubstituted 6-membered aryl, and ring B is a substituted or unsubstituted 5-membered alicyclic or heteroalicyclic. In some embodiments, ring B is a substituted or unsubstituted 6-membered aryl, and ring A is a substituted or unsubstituted 5-membered alicyclic or heteroalicyclic. In some embodiments of the present invention, both ring A and ring B are each a substituted or unsubstituted 6-membered alicyclic or heteroalicyclic.

When substituted, each or ring A and ring B can exhibit one or more of the following exemplary substituents, which include, without limitation, halo, cyano, nitrile, nitro, C₁₋₆ alkyl, C₁₋₆ alkenyl, alkynyl, germinal C₁₋₆ alkanes, benzyl, aryl, heteroaryl, alkoxy, aryloxy, carbonyl, carboxyl, carboxylate (ester), amide, alkylsulfonyl, heterobicycle, bi-phenyl, substituted bi-phenyl, bi-aryl, and substituted bi-aryl, provided that the presence of the substituent is chemically feasible.

In some embodiments of the present invention, the substituents are selected according to the bioavailability that is observed in the resulting compound, using the criteria of octanol-water partition coefficient (Log P_{ow}) value. Hence, according to some embodiments of the present invention, the compound is characterized by exhibiting a Log P_{ow} value that ranges from −1 to 6, or from −0.5 to 5, or from 0 to 5, or from 0 to 4. The selection of particular substituents and also their position on ring A or ring B, can be carried out by using a Log P_{ow} prediction algorithm, also known as cLog P_{ow} (for additional information regarding cLog P_{ow} prediction, see e.g., Ghose, Arup K. et al., J. Phys. Chem. A, 1998, 102(21), pp. 1089-5639).

As demonstrated in the Examples section that follows below, the present inventors have reduced the present invention to practice by synthesizing a series of exemplary compounds, according to some embodiments of the present invention but without limiting the scope of the invention, which include the compounds presented in Table 2, and Table 3 below. According to preferred embodiments of the present invention, the compound provided herein is not one of the compounds disclosed in the Chang study (Chang, C.-F. et al., European Journal of Medicinal Chemistry, 2016, 124, pp. 186-199). According to preferred embodiments of the present invention, the compound provided herein is not one of the compounds disclosed in, for example, U.S. Patent Application No. 2005/0009876, JP 2011246389, WO 2014/130693, WO 2016/004272, WO 2016/010108, Lanning, B. R. et al., Nat Chem Biol., 2014, 10(9), pp. 760-767 and Zhao, Z. et al., J. Med. Chem., 2017, 60, pp. 2879-2889, and compounds such as (Z)-4-oxo-4-((3-(6-(phenylsulfonamido)-1H-indazol-3-yl)phenyl)amino)but-2-enoic acid, N-[3-oxo-3-[[3-(4,5,6,7-tetrahydro-1H-indazol-3-yl)phenyl]amino]propyl]-2-Propenamide, N-methyl-N-[2-oxo-2-[[3-(4,5,6,7-tetrahydro-1H-indazol-3-yl)phenyl]amino]ethyl]-2-Propenamide, and 3-[5-(4,5,6,7-tetrahydro-5-methyl-1H-pyrazolo[4,3-c]pyridin-3-yl)-3-pyridinyl]-2-Propenoic acid ethyl ester. According to some embodiments of the present invention, the compound provided herein is not one of the compounds disclosed in public prior to conceiving the present invention, which are hereby excluded from the scope of the invention. The contents of the aforementioned publications are incorporated by reference herein in their entirety.

Excluded from the scope of formula I as a novel compound, but not necessarily from the scope of other aspects of the present invention, such as uses and pharmaceutical composition, are the compounds enumerated in the Chang study, such as:

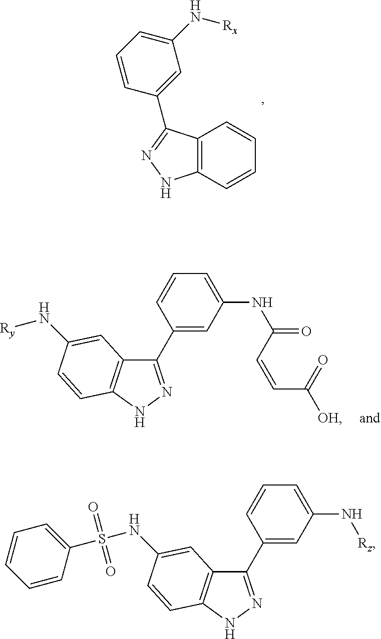

wherein:
Rₓ is selected from the group consisting of

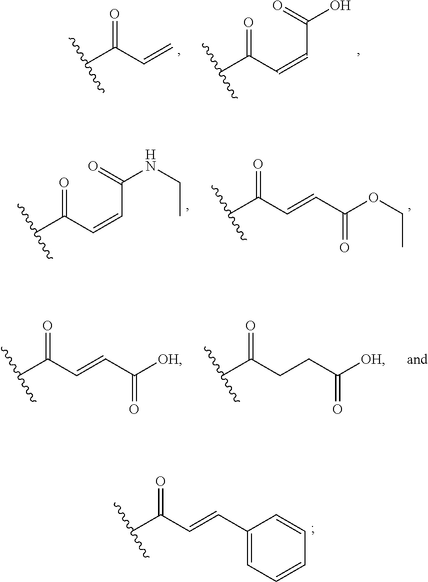

R_{y} is selected from the group consisting of PhNHCO, 4-Ph-PhSO₂, PhCO, 4-tBu-PhSO₂, PhCH₂, 4-OCH₃-PhSO₂, PhSO₂, 4-NO₂-PhSO₂, 4-CH₃-PhSO₂, and 4-F-PhSO₂; and
R_{z} is selected from the group consisting of

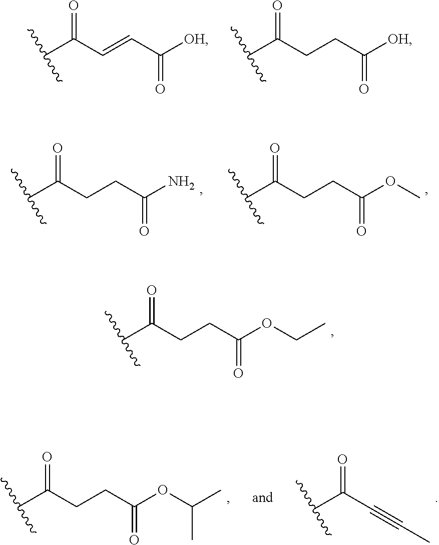

While reducing the present invention to practice, the following compounds were identified as potential leads compounds for the development of optimal covalent MKK7 inhibitors:

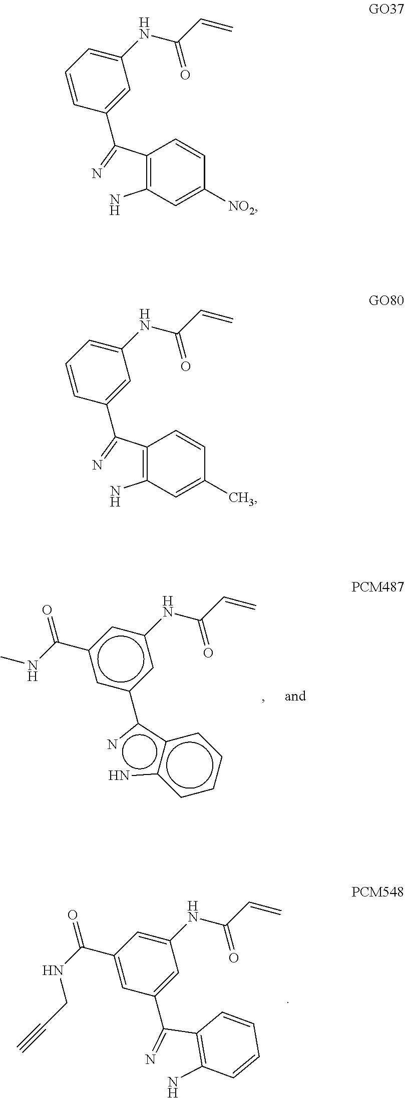

Hence, according to some embodiments of the present invention, the compound exhibits an electron withdrawing group at position corresponding to R₃ in formula I. Further alternatively, the compound according to some embodiments of the present invention, exhibits a substituent at position corresponding to R₆ in formula I.

Process of Preparing the Compounds:

According to some embodiments of an aspect of the present invention, there is provided a process of preparing the compounds presented herein, which is effected essentially as described in the Examples section hereinbelow.

Uses and Method of Treatment:

The present invention also provides uses of the compounds presented herein in a method of treating diseases, disorders, syndromes, or medical conditions, which are associated with c-Jun-NH₂-terminal kinase (JNK) pathway regulation, or more specifically, for treating medical conditions associated with MKK7. In some embodiments, the compound that is used as an active ingredient, is represented by formula I and as further described hereinabove, whereas the scope of formula I is contemplated with or without the proviso referring to the compounds presented in the Chang study, namely in the aspect of a method of treatment a disease, medical condition or disorder associated with human MKK7, the scope of the compounds may include compounds that were described elsewhere, as active ingredients for indications that are not associated with human MKK7. For example, the exemplary compound GO32, which has been described in the Chang study as an inhibitor of Aurora kinase, is contemplated in the context of the present invention as an active ingredient in a method of treatment and/or a pharmaceutical composition, indicated for diseases, medical conditions and disorders associated with MKK7. As can be seen in the Examples section below, GO32 is a far more potent inhibitor of MKK7 that any Aurora kinase, presumably due to the design paradigm of forming a covalent bond with a side chain of an amino acid residue in or near the active site of MKK7 (e.g., Cys218), which is not present in any Aurora kinase.

For example, the present invention provides a method of treating the above conditions in a subject in need thereof, which includes administering to the subject a therapeutically effective amount of one or more of the compounds presented herein, or a pharmaceutical composition that includes one or more of the compounds presented herein and a pharmaceutically acceptable carrier, thereby preventing or treating the aforementioned condition. Examples of routes of administration of the compounds presented herein include oral or parenteral, e.g., intravenous, intradermal, transdermal (topical), transmucosal, or administration by inhalation, and/or rectal administration, whereas each possibility represents a separate embodiment of the present invention.

In another aspect, the present invention relates to the use of the compounds presented herein for the manufacture of a medicament beneficial for the treatment of a medical condition, disease or disorder associated with c-Jun-NH₂-terminal kinase (JNK) pathway regulation. In some embodiments, the compound is used for the manufacture of a medicament, is represented by formula I and as further described hereinabove, whereas the scope of formula I is contemplated with or without the proviso referring to the compounds presented in the Chang study.

Diseases, disorders, syndromes, or medical conditions, which are associated with c-Jun-NH₂-terminal kinase (JNK) pathway regulation, include, without limitation and without being bound by any particular theory, Parkinson's disease, Alzheimer's disease, Pick's disease, Crohn's disease, Behcet's disease, stroke, coronary artery disease, heart failure, abdominal aortic aneurysm, noonan syndrome, chronic hepatitis C virus infection, acute liver injury, non-alcoholic fatty liver disease, asthma, chronic obstructive pulmonary disease, amyotrophic lateral sclerosis, inflammatory bowel disease, polyglutamine disease, auditory hair cell degeneration, rheumatoid arthritis, systemic lupus eryththematosus, celiac disease, colorectal cancer, retinoblastoma, melanoma, breast carcinoma, ovarian cancer, obesity, insulin resistant, progressive supranuclear palsy, corticobasal degeneration, argyrophilic grain disease, familial frontotemporal dementia, and type 2 diabetes.

According to some embodiments, the disease, disorder, syndrome, or medical condition is treatable based on the inhibition of human MKK7 enzyme, which exhibits a cysteine at position 218. In some embodiments, the disease, disorder, syndrome, or medical condition is not diabetes, inflammation or cancer.

As used herein, the term “administering” refers to bringing in contact with a compound of the present invention. Administration can be accomplished to cells or tissue cultures, or to living organisms, for example humans. In one embodiment, the present invention encompasses administering the compounds of the present invention to a human subject.

A “therapeutic” treatment is a treatment administered to a subject who exhibits signs of pathology for the purpose of diminishing or eliminating those signs. A “therapeutically effective amount” is that amount of compound which is sufficient to provide a beneficial effect to the subject to which the compound is administered.

“Preventing” and “prevent” means avoiding the onset of a clinically evident disease progression altogether or slowing the onset of a pre-clinically evident stage of a disease in individuals at risk. Prevention includes prophylactic treatment of those at risk of developing a disease.

Methods of treating a disease according to the invention may include administration of the pharmaceutical compositions or medicaments comprising the compounds presented herein as a single active agent, or in combination with additional methods of treatment. In some embodiments, the one or more additional agents may be added to the composition. The methods of treatment of the invention may be in parallel to, prior to, or following additional methods of treatment.

A Pharmaceutical Composition:

The compound, according to embodiments of the present invention, is a selective covalent inhibitor of MKK7, and can therefore be used also as an active ingredient in a pharmaceutical composition for the treatment of a medical condition, a disease or a disorder associated with c-Jun-NH₂-terminal kinase (JNK) pathway regulation, or more specifically, for treating medical conditions associated with MKK7.

According to an additional aspect of embodiments of the present invention, there is provided a pharmaceutical composition that includes, as an active ingredient, one or more of the compounds presented herein. In some embodiments, the compound that is used as an active ingredient in a pharmaceutical composition, is represented by formula I and as further described hereinabove, whereas the scope of formula I is contemplated with or without the proviso referring to the compounds presented in the Chang study.

The present invention provides, in some embodiments, pharmaceutical compositions comprising, as an active ingredient, the compounds presented herein and a pharmaceutically acceptable carrier. The term “pharmaceutically acceptable carrier” refers to a vehicle which delivers the active components to the intended target and which does not cause harm to humans or other recipient organisms. As used herein, “pharmaceutical” will be understood to encompass both human and animal pharmaceuticals. Useful carriers include, for example, water, acetone, ethanol, ethylene glycol, propylene glycol, butane-1, 3-diol, isopropyl myristate, isopropyl palmitate, or mineral oil. Use of detergents such as n-octyl-β-D-glucopyranoside (OGP) is also contemplated. Methodology and components for formulation of pharmaceutical compositions are well known, and can be found, for example, in Remington's Pharmaceutical Sciences, Eighteenth Edition, A. R. Gennaro, Ed., Mack Publishing Co. Easton Pa., 1990, the contents of which are incorporated by reference in their entirety.

Typically, pharmaceutical composition are formulated in any form appropriate to the mode of administration, for example, solutions, colloidal dispersions, emulsions (oil-in-water or water-in-oil), suspensions, creams, lotions, gels, foams, sprays, aerosol, ointment, tablets, suppositories, and the like. In some embodiments, the pharmaceutical compositions of the present invention are formulated for aerosol administration for inhalation by a subject in need thereof.

A therapeutically effective amount of the compounds presented herein in a pharmaceutical composition, according to some embodiments of the present invention, is an amount that when administered to a subject, is capable of preventing or ameliorating an infection, e.g., bacterial infection, or one or more symptoms thereof. The effective amount of an agent or composition of the present invention administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions of the present invention can also be administered in combination with one or more additional therapeutic compounds. Precise amounts of active ingredient required to be administered depend on the judgment of the practitioner and are peculiar to each individual.

Typically, the therapeutic agent will be administered as a pharmaceutical formulation that includes the therapeutic agent and any pharmaceutically acceptable adjuvants, carriers, excipients, and/or stabilizers, and can be in solid or liquid form, such as tablets, capsules, powders, solutions, suspensions, or emulsions. The compositions preferably contain from about 0.01 to about 99 weight percent, more preferably from about 2 to about 60 weight percent, of therapeutic agent together with the adjuvants, carriers and/or excipients. In some embodiments, an effective amount ranges from about 0.001 mg/kg to about 500 mg/kg body weight of the subject. In some embodiments, the effective amount of the agent ranges from about 0.05 mg/kg to about 30 mg/kg, from about 0.1 mg/kg to about 30 mg/kg, from about 1 mg/kg to about 25 mg/kg, 30 from about 1 mg/kg to about 20 mg/kg, or from about 1 or 2 mg/kg to about 15 mg/kg.

In some embodiments, the composition of the invention is administered by intranasal or intraoral administration, using appropriate solutions, such as nasal solutions or sprays, aerosols or inhalants. Nasal solutions are usually aqueous solutions designed to be administered to the nasal passages in drops or sprays. Typically, nasal solutions are prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations, and appropriate drug stabilizers, if required, may be included in the formulation. Various commercial nasal and oral preparations for inhalation, aerosols and sprays are known and include, for example, antibiotics and antihistamines and are used for asthma prophylaxis.

For intranasal or intraoral administration the composition of the invention is provided in a solution suitable for expelling the pharmaceutical dose in the form of a spray, wherein a therapeutic quantity of the pharmaceutical composition is contained within a reservoir of an apparatus for nasal or intraoral administration. The apparatus may comprise a pump spray device in which the means for expelling a dose comprises a metering pump. Alternatively, the apparatus comprises a pressurized spray device, in which the means for expelling a dose comprises a metering valve and the pharmaceutical composition further comprises a conventional propellant. Suitable propellants include one or mixture of chlorofluorocarbons, such as dichlorodifluoromethane, trichlorofluoromethane, dichloro-tetrafluoroethane, hydrofluorocarbons, such as 1,1,1,2-tetrafluoroethane (HFC-134a) and 1,1,1,2,3,3,3-heptafluoropropane (HFC-227) or carbon dioxide. Suitable pressurized spray devices are well known in the art and include those disclosed in, inter alia, WO 92/11190, U.S. Pat. Nos. 4,819,834, 4,407,481 and WO 97/09034, when adapted for producing a nasal spray, rather than an aerosol for inhalation, or a sublingual spray. The contents of the aforementioned publications are incorporated by reference herein in their entirety. Suitable nasal pump spray devices include the VP50, VP70 and VP100 models available from Valois S.A. in Marly Le Roi, France and the 50, 70 and 100 μl nasal pump sprays available from Pfeiffer GmbH in Radolfzell, Germany, although other models and sizes can be employed. In the aforementioned embodiments, a pharmaceutical dose or dose unit in accordance with the invention can be present within the metering chamber of the metering pump or valve.

It is expected that during the life of a patent maturing from this application many relevant covalent MKK7 inhibitors will be developed and the scope of the term covalent MKK7 inhibitors is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the phrases “substantially devoid of” and/or “essentially devoid of” in the context of a certain substance, refer to a composition that is totally devoid of this substance or includes less than about 5, 1, 0.5 or 0.1 percent of the substance by total weight or volume of the composition. Alternatively, the phrases “substantially devoid of” and/or “essentially devoid of” in the context of a process, a method, a property or a characteristic, refer to a process, a composition, a structure or an article that is totally devoid of a certain process/method step, or a certain property or a certain characteristic, or a process/method wherein the certain process/method step is effected at less than about 5, 1, 0.5 or 0.1 percent compared to a given standard process/method, or property or a characteristic characterized by less than about 5, 1, 0.5 or 0.1 percent of the property or characteristic, compared to a given standard.

The term “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The words “optionally” or “alternatively” are used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the terms “process” and “method” refer to manners, means, techniques and procedures for accomplishing a given task including, but not limited to, those manners, means, techniques and procedures either known to, or readily developed from known manners, means, techniques and procedures by practitioners of the chemical, material, mechanical, computational and digital arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein, the term “alkyl” describes an aliphatic hydrocarbon including straight chain and branched chain groups. The alkyl group may exhibit 1 to 20 carbon atoms, and preferably 8-20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. The alkyl can be substituted or unsubstituted, and/or branched or unbranched (linear). When substituted, the substituent, referred to herein as R, can be, for example, an alkyl, an alkenyl, an alkynyl, a cycloalkyl, an aryl, a heteroaryl, a halo, a hydroxy, an alkoxy and a hydroxyalkyl as these terms are defined herein. In some embodiments of the present invention, the term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be branched or unbranched (linear), substituted or unsubstituted by one or more substituents, as described herein.

The term “alkynyl”, as defined herein, is an unsaturated alkyl having at least two carbon atoms and at least one carbon-carbon triple bond. The alkynyl may be branched or unbranched (linear), and/or substituted or unsubstituted by one or more substituents, as described herein.

The terms “alicyclic” and “cycloalkyl”, refer to an all-carbon monocyclic or fused ring (i.e., rings which share an adjacent pair of carbon atoms), branched or unbranched group containing 3 or more carbon atoms where one or more of the rings does not have a completely conjugated pi-electron system, and may further be substituted or unsubstituted. The cycloalkyl can be substituted or unsubstituted by one or more substituents, as described herein.

The term “heteroalicyclic”, refer to a monocyclic or fused ring (i.e., rings which share an adjacent pair of atoms) group having in the ring(s) one or more non-carbon atoms, such as, for example, nitrogen, oxygen and sulfur and, where one or more of the rings does not have a completely conjugated pi-electron system. The heteroalicyclic can be substituted or unsubstituted by one or more substituents, as described herein.

The term “aryl” describes an all-carbon aromatic monocyclic or fused-ring polycyclic (i.e., rings which share adjacent pairs of carbon atoms) groups having a completely conjugated pi-electron system. The aryl group may be substituted or unsubstituted. Substituted aryl may have one or more substituents as described for alkyl herein.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system.

Representative examples of heteroaryls include, without limitation, furane, imidazole, indole, isoquinoline, oxazole, purine, pyrazole, pyridine, pyrimidine, pyrrole, quinoline, thiazole, thiophene, triazine, triazole and the like. The heteroaryl group may be substituted or unsubstituted as described for alkyl herein.

The term “halo” refers to —F, —Cl, —Br or —I.

The term “hydroxy”, as used herein, refers to an —OH group.

The terms “alkoxy” and “hydroxyalkyl” refer to a —OR group, wherein R is alkyl as described hereinabove.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental and/or calculated support in the following examples.

### EXAMPLES

Reference is now made to the following examples, which together with the above descriptions illustrate some embodiments of the invention in a non limiting fashion.

### Example 1

### Docking

Docking Method:

DOCKovalent [London, N. et al., “Covalent docking of large libraries for the discovery of chemical probes”, Nat Chem Biol, 2014, 10(12), pp. 1066-72] was used to screen a virtual library of 117,667 acrylamides against residue Cys218 of MKK7, situated near the active site of the enzyme. This library contained commercially available building blocks with a suitable free amine (primary or secondary, aliphatic or aromatic), that was virtually converted to the corresponding acrylamide.

Initial Hits:

Based on visual inspection of the top 500 predictions, 10 compounds were selected for synthesis and testing (see, Table 1). The “Docking rank” is given in its respective run, whereas four virtual libraries of acrylamides were docked separately, based on the amine precursor for the acrylamide (primary or secondary aliphatic amines, primary or secondary aromatic amines). In vitro kinase activity assay was carried out by Nanosyn, as described below.

TABLE 1

| Compound | | DOCKing | In vitro IC₅₀ |
|---|---|---|---|
| No. | Compound Structure | rank | (μM) |
| GO1 | 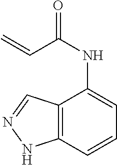 | 274 | >10 |
| GO2 | 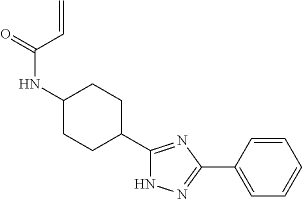 [SMILES: C=CC(=O)NC1CCC(c2nc(-c3ccccc3)n[nH]2)CC1] | 39 | >10 |
| GO4 | 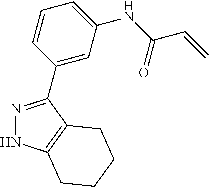 [SMILES: C=CC(=O)Nc1cccc(-c2n[nH]c3c2CCCC3)c1] | 300 | 0.011 |
| GO5 | 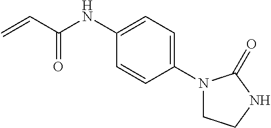 [SMILES: C=CC(=O)Nc1ccc(N2CCNC2=O)cc1] | 305 | 0.502 |
| GO6 | 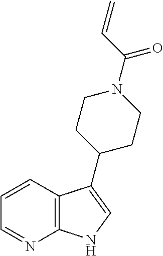 [SMILES: C=CC(=O)N1CCC(c2c[nH]c3ncccc23)CC1] | 16 | >10 |
| GO7 | 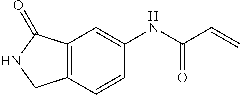 | 95 | 0.873 |
| GO8 | 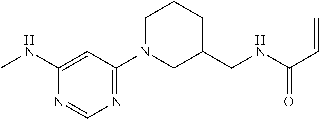 [SMILES: C=CC(=O)NCC1CCCN(c2cc(NC)ncn2)C1] | 49 | >10 |
| GO9 | 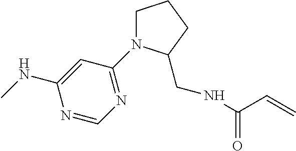 [SMILES: C=CC(=O)NCC1CCCN1c1cc(NC)ncn1] | 37 | >10 |
| GO10 | 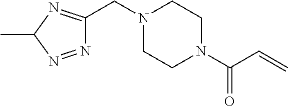 [SMILES: C=CC(=O)N1CCN(CC2=NC(C)N=N2)CC1] | 141 | >10 |
| GO14 | 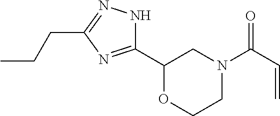 [SMILES: C=CC(=O)N1CCOC(c2nc(CCC)n[nH]2)C1] | 41 | >10 |

Of the top 10 hits, three compounds showed inhibition of MKK7 in an in vitro kinase activity assay with IC₅₀ of 0.011 μM, 0.502 μM and 0.873 μM, and GO4 was selected for further optimization.

### Example 2

### Synthesis

Based on the predicted docking model, an exemplary series of GO4 analogs was designed and synthesized (see, Table 2), whereas the in vitro kinase activity assay carried out by Nanosyn (see below), and the EC₅₀ was an in-cell western assay (ICW) assessment of pJNK inhibition.

TABLE 2

| | | MKK7 | MKK4 | EC₅₀ | EC₅₀ |
|---|---|---|---|---|---|
| Compound | | IC₅₀ in | IC₅₀ in | HEK293 | 3T3 WT |
| No. | Compound Structure | viro (μM) | vitro (μM) | (μM) | (μM) |
| GO4 | 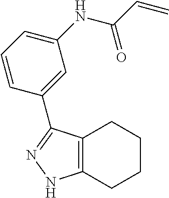 [SMILES: C=CC(=O)Nc1cccc(-c2n[nH]c3c2CCCC3)c1] | 0.011 | 4.13 | >10 2.9 5.2 | >10 |
| GO29 | 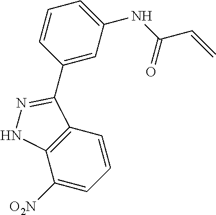 [SMILES: C=CC(=O)Nc1cccc(-c2n[nH]c3c([N+](=O)[O-])cccc23)c1] | 0.027 | | >10 | |
| GO32 | 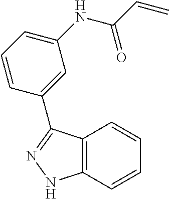 [SMILES: C=CC(=O)Nc1cccc(-c2n[nH]c3ccccc23)c1] | 0.006 | 4.15 | 0.38 2.06 | 4.3 5.9 15.6 4.4 4.2 4.1 3.2 |
| GO37 | 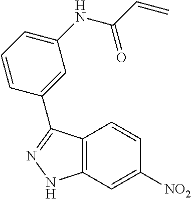 [SMILES: C=CC(=O)Nc1cccc(-c2n[nH]c3cc([N+](=O)[O-])ccc23)c1] | 0.014 | >10 | 0.73 | 4.2 4.2 2.2 10.4 |
| GO49 | 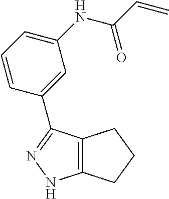 [SMILES: C=CC(=O)Nc1cccc(-c2n[nH]c3c2CCC3)c1] | 0.073 | | | |
| GO54 | 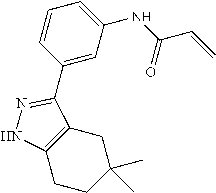 [SMILES: C=CC(=O)Nc1cccc(-c2n[nH]c3c2CC(C)(C)CC3)c1] | 0.010 | | 3.3 | |
| GO55 | 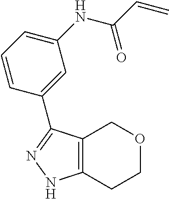 [SMILES: C=CC(=O)Nc1cccc(-c2n[nH]c3c2COCC3)c1] | 0.064 | | >10 | |
| GO61 | 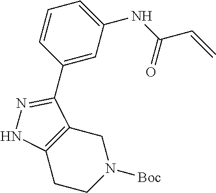 [SMILES: C=CC(=O)Nc1cccc(-c2n[nH]c3c2CN(C(=O)OC(C)(C)C)CC3)c1] | 0.193 | | >10 | |
| GO64 | 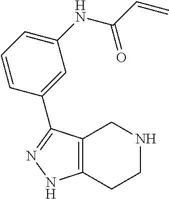 [SMILES: C=CC(=O)Nc1cccc(-c2n[nH]c3c2CNCC3)c1] | 2.462 | | >10 | |
| GO65 | 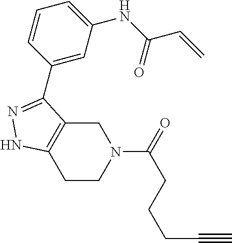 [SMILES: C#CCCCC(=O)N1CCc2[nH]nc(-c3cccc(NC(=O)C=C)c3)c2C1] | 0.030 | | 1.9 >10 | |
| GO72 | 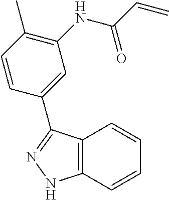 [SMILES: C=CC(=O)Nc1cc(-c2n[nH]c3ccccc23)ccc1C] | 0.026 | | 4.2 | |
| GO73 | 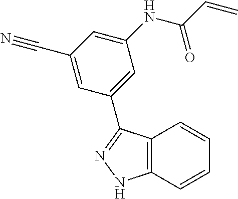 [SMILES: C=CC(=O)Nc1cc(C#N)cc(-c2n[nH]c3ccccc23)c1] | 0.004 | | 2.3 0.4 | |
| GO74 | 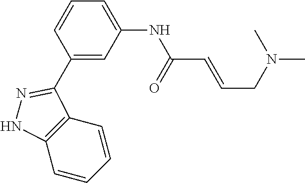 [SMILES: CN(C)C/C=C/C(=O)Nc1cccc(-c2n[nH]c3ccccc23)c1] | 0.267 | | | |
| GO80 | 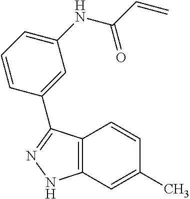 [SMILES: C=CC(=O)Nc1cccc(-c2n[nH]c3cc(C)ccc23)c1] | 0.005 | 7.81 | 1.3 0.8 | 3.4 2.3 8.4 3.8 7.3 |
| GO81 | 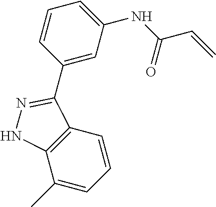 [SMILES: C=CC(=O)Nc1cccc(-c2n[nH]c3c(C)cccc23)c1] | 0.812 | | | |
| GO83 | 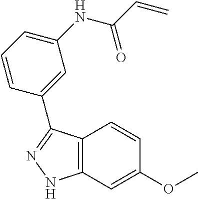 [SMILES: C=CC(=O)Nc1cccc(-c2n[nH]c3cc(OC)ccc23)c1] | 0.011 | | | |
| GO88 | 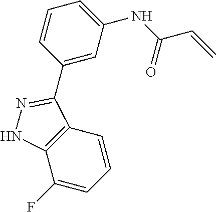 [SMILES: C=CC(=O)Nc1cccc(-c2n[nH]c3c(F)cccc23)c1] | 0.004 | | 1.8 | |
| GO89 | 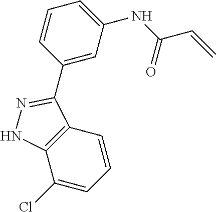 [SMILES: C=CC(=O)Nc1cccc(-c2n[nH]c3c(Cl)cccc23)c1] | 0.047 | | | |
| GO98 | 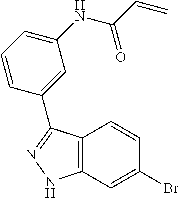 [SMILES: C=CC(=O)Nc1cccc(-c2n[nH]c3cc(Br)ccc23)c1] | 0.010 | >10 | 1.0 0.3 | |
| GO101 | 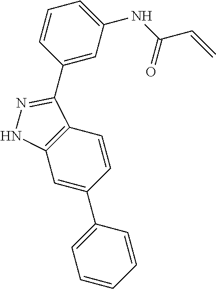 [SMILES: C=CC(=O)Nc1cccc(-c2n[nH]c3cc(-c4ccccc4)ccc23)c1] | 0.011 | | 1.0 | |
| GO106 | 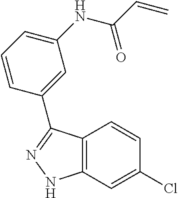 [SMILES: C=CC(=O)Nc1cccc(-c2n[nH]c3cc(Cl)ccc23)c1] | 0.005 | | 3.0 | |
| GO108 | 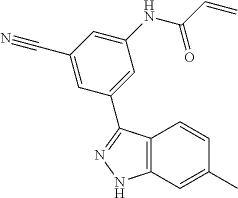 [SMILES: C=CC(=O)Nc1cc(C#N)cc(-c2n[nH]c3cc(C)ccc23)c1] | 0.002 | >10 | 3.7 2.3 | |

General:

All reagents and solvents used for the synthesis were purchased from Sigma-Aldrich, Merck and Acros. Chemical building blocks were purchased from Enamine and MolPort chemical suppliers. Commercial reagents were used for synthesis without further purification. Flash chromatography was performed using Merck Silica gel Kieselgel 60 (0.04-0.06 mm) or by atomized CombiFlash® Systems (Teledyne Isco, USA) with RediSep Rf Normal-phase Flash Columns. Purification of the final compounds was performed using preparative HPLC; Waters Prep 2545 Preparative Chromatography System, with UVNis detector 2489, using XBridge® Prep C18 10 μm 10×250 mm Column (PN: 186003891, SN:161I3608512502). Reaction progress and compounds' purity were monitored by Waters UPLC-MS system: Acquity UPLC® H class with PDA detector, and using Acquity UPLC® BEH C18 1.7 μm 2.1×50 mm Column (PN:186002350, SN 02703533825836). MS-system: Waters, SQ detector 2. ¹H and ¹³C NMR spectra were recorded on a Bruker Avance −300 MHz, 400 MHz and 500 MHz spectrometer, equipped with QNP probe. Chemical shifts are reported in ppm on the 8 scale down field from TMS. All J values are given in Hertz. High resolution electron-spray mass spectrometry (HR-MS) was performed on a Xevo G2-XS QTOF Mass Spectrometer (Waters Corporation, USA).

Design of Synthesis:

Two main strategies were applied to prepare the exemplary compounds, according to some embodiments of the present invention, the first is Synthetic Pathway A to generate 4,5,6,7-tetrahydro-1H-indazole template, and the second is Synthetic Pathway B to generate 1H-indazole template.

Synthetic Pathway A: Synthesis of 3-phenyl-4,5,6,7-tetrahydro-1H-indazole Template:

Compounds bearing 3-phenyl-4,5,6,7-tetrahydro-1H-indazole template were prepared as described in Scheme 1 below. Commercially available cyclohexanone or its derivatives were converted to enamine intermediates using morpholine, the enamine was reacted further with acyl-chloride derivative (Alloc protected 3-aminobenzoyl chloride) which was prepared separately. Hydrolysis of the enamine provided 1,3-diketone. Cyclization of the 1,3-diketones to pyrazoles was obtained by the addition of hydrazine. Deprotection of Alloc group form 4,5,6,7-tetrahydro-1H-indazole template bearing Alloc-N-aryl provided the free amine precursor for acrylation.

In Scheme 1, the reaction conditions are: (a) Allyl chloroformate, 4 M NaOH (aq.), dioxane, 0° C. to RT; (b) Oxalyl chloride, DCM, DMF, 0° C. to RT; (c) morpholine, p-toluenesulfonic acid, toluene, reflux; (d) CHCl₃, Et₃N, 0° C. to RT; HCl, H₂O, RT; (e) NH₂NH₂—H₂O, B(OCH₃)₃, CHCl₃. RT; (f) phenylsilane, Pd(PPh₃)₄, CH₂Cl₂, RT.

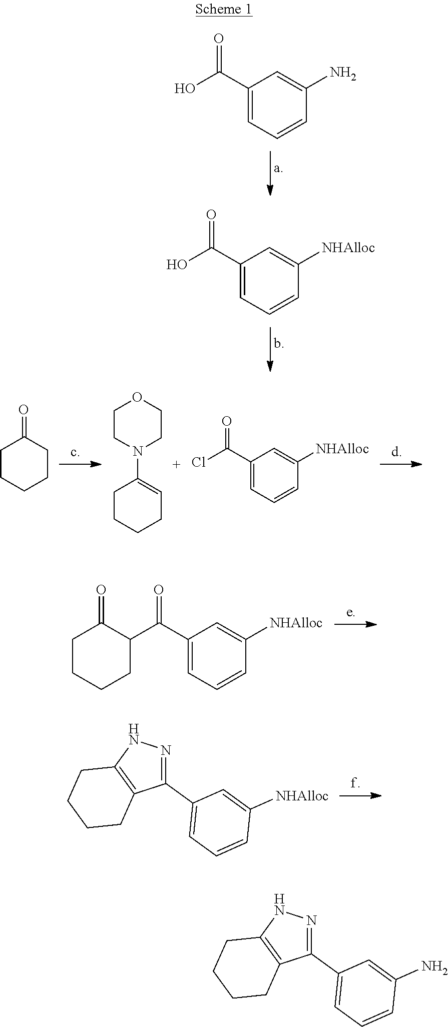

Acyl-Halide Preparation:

Prior to acyl-halide generation, the free amine of 3-aminobenzoic acid was protected with Alloc protecting group as following: 3-aminobenzoic acid (2 gr, 0.014 mol, 1 eq.) was dissolved in 4M NaOH (10 mL) and dioxane (4 mL) and cooled over ice bath. Allyl chloroformate (2 mL, 0.019 mol, 1.3 eq.) was dissolved in 4M NaOH (5 mL) and added in several portions to the reaction mixture. If required the pH of the mixture was adjusted to 10 with a solution of 4M NaOH. The reaction was left to stir at RT overnight, diluted with H₂O, acidified with saturated citric acid to neutral pH, and extracted with EtOAc. The organic layer was dried over Na₂SO₄ and EtOAc was evaporated to provide the Alloc protected compound. 3-Alloc-aminobenzoic acid (0.3 gr, 1.35 mmol, 1 eq.) was dissolved in dry DCM (10 mL) and cooled over ice bath. Oxalyl chloride (150 μL, 1.76 mmol, 1.3 eq.) was added under inert atmosphere via syringe, following addition of 2-3 drops of dry DMF. The reaction was warmed to RT and after 1 hour of stirring additional portion of oxalyl chloride was added (10 μL, 0.1 mmol, 0.08 eq.) the reaction was stirred for additional 2-3 hours. DCM was evaporated to provide allyl-(3-(chlorocarbonyl)phenyl)carbamate as white solid.

General Procedure for Enamine Preparation:

Cyclohexanone or similar derivative (0.019 mol, 1 eq.) was dissolved in toluene (10 mL), to the mixture were added morpholine (0.018 mol, 0.95 eq.) and p-toluenesulfonic acid (cat.; 0.01 eq.). The reaction flask was fitted with a Dean-Stark apparatus and the mixture was refluxed until no additional separation of water was observed (2-3 hours). The reaction was concentrated in vacuo, and the oily crude product was used without additional purification.

General Procedure for Acylation of Enamines and Formation of 1,3-diketone:

An appropriate enamine (3.76 mmol, 1 eq.) was dissolved in anhydrous chloroform (5 mL) and was added in few portions to a solution of allyl-(3-(chlorocarbonyl)phenyl)carbamate (3.2 mmol, 0.85 eq) in anhydrous chloroform (3.2 mL) under inert atmosphere and over ice bath. Next, Et₃N (3.76 mmol, 1 eq.) was added dropwise to the reaction mixture. The reaction was stirred at RT for 3.5 hours, and monitored by UPLC till consumption of the acyl-chloride. After all acyl-chloride has reacted, water (15 mL) and concentrated HCl solution (1.5 mL) were added to the reaction mixture and stirred for 2 hours till formation of the appropriate 1,3-diketone. The mixture was extracted with EtOAc and washed with saturated solution of citric acid. The organic layer was dried over Na₂SO₄ and concentrated in vacuo to yield the crude residue. The residue was purified by silica gel chromatography (gradient, hexane to EtOAC) to yield the product.

General Procedure for 4,5,6,7-tetrahydro-1H-indazole Preparation:

An appropriate 1,3-diketone (1.32 mmol, 1 eq.) was dissolved in anhydrous chloroform (15 mL). To the reaction mixture were added: hydrazine monohydrate (1.58 mmol, 1.2 eq.) and trimethyl borate (in excess, 5 mL). The reaction was stirred for 20-30 min at RT, and the product formation was monitored by UPLC. The reaction was quenched by addition of water (1-2 mL). Chloroform was evaporated and the crude was re-dissolved in EtOAc and extracted twice with water. The organic layer was dried over Na₂SO₄ and concentrated in vacuo to yield the crude residue. The residue was purified by silica gel chromatography (gradient, DCM to EtOAC) to yield the product.

General Procedure for Alloc Deprotection:

Alloc protected compound (Alloc-N-aryl; 0.18 mmol, 1 eq.) was dissolved in anhydrous DCM (8 mL), phenylsilane (0.45 mmol, 2.5 eq.) was added to the mixture. The solution was degassed by bubbling argon through the solvent. After 5 min Pd(PPh₃)₄ catalyst (0.018 mmol, 0.1 eq) was added under a strong flow of argon. After the addition, the argon flow was stopped, and the flask was covered with aluminum foil. The reaction mixture was stirred for 3 hours at RT, under inert atmosphere. After the reaction was finished, the solvent was concentrated in vacuo to provide the crude residue, which was purified by silica gel chromatography (gradient: DCM to EtOAC)

Synthetic Pathway B: Synthesis of 3-phenyl-1H-indazole Based Compounds:

Compounds bearing the 3-phenyl-1H-indazole template were prepared as described in Scheme 2 below. Suzuki-Miyaura cross coupling reaction between commercially available 3-acyl-1H-indazole or its derivative with 3-(N-Boc-amino)phenylboronic acid (which was prepared by bocylation of 3-aminophenylboronic acid) generated the required structural template, i.e., 3-phenyl-1H-indazole. Acidolysis of Boc provided the target amine for acrylation. In Scheme 2, the reaction conditions are: (a) Boc₂O, NEt₃, THF/H₂O, 0° C. to RT; (b) Pd(PPh₃)₄, phenylsilane, CH₂Cl₂ (c) 50% TFA in CH₂Cl₂, triisopropylsilane, 0° C. to RT.

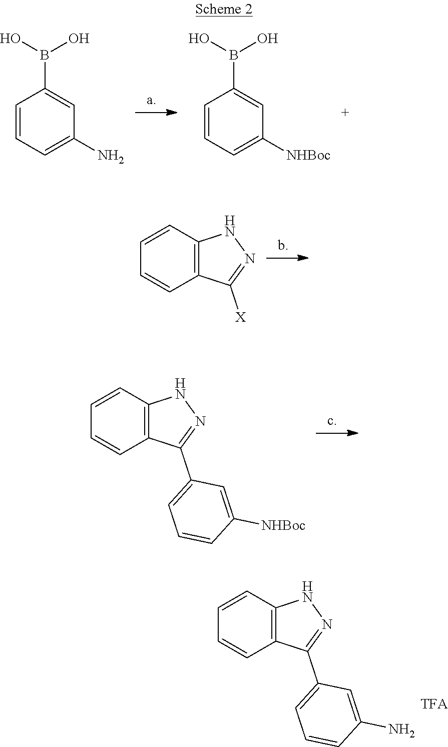

General Procedure for Bocylation of 3-aminophenylboronic Acid:

3-aminophenylboronic acid monohydrate or its derivatives were bocylated as described elsewhere [WO 2008/134036].

General Procedure for Suzuki Coupling:

An appropriate indazole substituted with halide (bromide or iodide; 0.084 mmol, 1 eq.) was dissolved in dioxane (5 mL). To the reaction mixture were added 3-(N-Boc-amino)phenylboronic acid (0.12 mmol, 1.5 eq.), and K₂CO₃ (0.16 mmol, 2 eq.) dissolved in water (1 mL). The solution was degassed by bubbling argon through the solvent. After 20 minutes Pd(PPh₃)₄ catalyst (0.006 mmol, 0.07 eq.) was added, under a strong flow of argon. After the addition, the argon flow was stopped, and the reaction mixture was stirred for 5 hours at 100° C. under inert atmosphere, then was left to stir at RT for 1-2 days, until HPLC showed consumption of the starting indazole. The reaction mixture was then cooled, diluted with EtOAc (50 mL), and extracted with saturated citric acid. The crude residue afforded was purified by silica gel chromatography (gradient: DCM to EtOAC).

General Procedure for Boc Acidolysis:

Boc protected compound (1.1 mmol, 1 eq.) was cooled to 0° C. over ice bath, and dissolved in DCM: TFA (2:1, tot. 18 mL) following addition of a drop of Triisopropylsilane. The reaction mixture was stirred at RT for 30-60 min; the solvent was removed in vacuo to yield the product.

Synthesis of Substituted N□Aryl Acrylamide Derivatives:

The synthesis of N-aryl acrylamides is presented in Scheme 3 below, and involves one-step condensation of acrylate chloride and corresponding N-aryl in dry CH₂Cl₂ in the presence of NEt₃. The reaction conditions in Scheme 3 are: Acryloyl chloride, DMF:ACN, base; R=1H-indazole, 4,5,6,7-tetrahydro-1H-indazole or commercially available heterocycle.

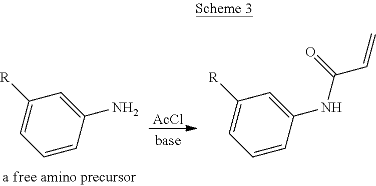

General Procedure for Acrylation:

Amine bearing precursor (commercially available or alternatively prepared in the lab; 1 eq.) was dissolved in mixture of anhydrous DMF and ACN, the mixture was cooled to 0° C. over an ice-bath and AcCl (0.8-1.2 eq.) was added to the mixture. The pH of the reaction mixture was adjusted to 9 using base (DIPEA or Et₃N), and the reaction was left to stir over ice and then at RT till product was formed. Upon reaction completion, the solvent was evaporated and the residue was suspended in EtOAc and washed twice with 3 M HCl, once with solution of saturated NaHCO₃ and once with brine. The organic extract was dried over Na₂SO₄ and evaporated in vacuo to yield the crude product, which were purified by preparative HPLC over C18 column, using buffers: 0.1% TFA in DDW and 0.1% TFA in ACN. The products were eluted with gradient of 99% DDW to 99% ACN. Exceptions in the procedure are described below.

Synthetic Procedures and Characterization Data for Exemplary Acrylamide Bearing Compounds:

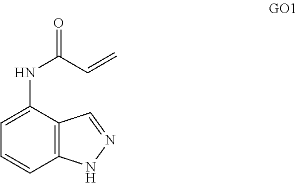

Following general acrylation procedure, N-aryl precursor (available from a commercial source; 0.02 g, 0.15 mmol, 1 eq.), AcCl (13.5 μL, 0.16 mmol, 1.1 eq.), DIPEA (till basic pH), were stirred in a mixture of DMF: ACN (1 mL: 1 mL) for 30 min. over ice-bath and at RT overnight.

The product was obtained as white powder.

¹H NMR (400 MHz, MeOD) δ ppm 5.84 (dd, J=10.2 Hz, 1.7 Hz, 1H), 6.44 (dd, J=17 Hz, 1.7 Hz, 1H), 6.65 (dd, J=17 Hz, 10.2 Hz, 1H), 7.4-7.34 (m, 2H), 7.66 (d, J=6.6 Hz, 1H), 8.24 (s, 1H).

HRMS: m/z, C₁₀H₁₀N₃O, required 188.0824 [M+H]⁺, found 188.0822 [M+H]⁺.

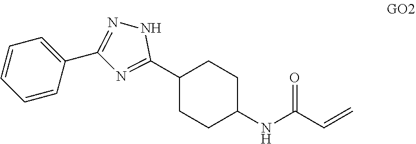 [SMILES: C=CC(=O)NC1CCC(c2nc(-c3ccccc3)n[nH]2)CC1]

Following general acrylation procedure, N-aryl precursor (available from a commercial source; 0.02 g, 0.08 mmol, 1 eq.), AcCl (7.5 μL, 0.09 mmol, 1.1 eq.), DIPEA (till basic pH), were stirred in a mixture of DMF: ACN (0.5 mL: 1 mL) for 30 min. over ice-bath and at RT for 2 hours.

The product was obtained as white powder.

¹H NMR (400 MHz, Acetone-D6) δ ppm 1.50-1.40 (m, 2H), 1.86-1.75 (m, 2H), 2.21-2.10 (m, 2H), 2.95-2.87 (m, 1H), 3.91-3.83 (m, 1H), 5.57 (dd, J=9, 3 Hz, 1H), 6.27-6.24 (m, 2H), 7.49-7.04 (m, 3H), 8.11 (m, 2H). ¹³C NMR (100 MHz, Acetone-D6): δ ppm 30.18, 32.05, 35.60, 47.68, 47.78, 124.45, 126.07, 128.52, 129.01, 130.90, 131.94, 131.98, 159.49, 162.33, 163.96. HRMS: m/z, C₁₇H₂₁N₄O, required 297.1715 [M+H]⁺, found 297.1707 [M+H]⁺, 319.1535 [M+Na]⁺, 615.3165 [2M+Na]⁺.

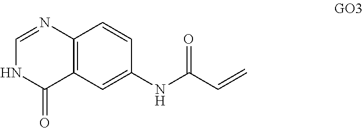 [SMILES: C=CC(=O)Nc1ccc2nc[nH]c(=O)c2c1]

In a modified protocol [Allen, C. E. et al., Organic Letters, 2015, 17(3), 458-460], N-aryl precursor (available from a commercial source) (0.02 g, 0.12 mmol, 1 eq.), AcCl (11.0 μL, 0.13 mmol, 1.1 eq), Amberlyst A26 (OH form) (270 mg) were stiffed in DMF (2 mL) for 5 min over ice-bath and 30 min at RT. The resin was filtrated off and the crude was injected to prep. HPLC, to provide the product as white solid powder.

¹H NMR (300 MHz, MeOD) δ ppm 5.84 (dd, J=8.6 Hz, 3.3 Hz, 1H), 6.47 (m, 2H), 7.71 (d, J=8.8 Hz 1H), 8.13 (dd, J=8.9 Hz, 2.5 Hz, 1H), 8.23 (s, 1H), 8.61 (d, J=2.5 Hz, 1H). HRMS: m/z, C₁₁HdN₃O2, required 216.0773 [M+H]⁺, found 216.0772 [M+H]⁺, 238.0592 [M+Na]⁺, 453.1298 [2M+Na]⁺.

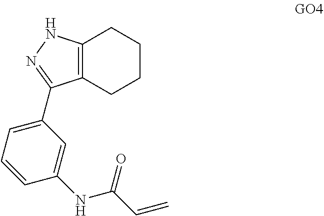 [SMILES: C=CC(=O)Nc1cccc(-c2n[nH]c3c2CCCC3)c1]

Following general acrylation procedure, N-aryl precursor (available from a commercial source; 0.02 g, 0.094 mmol), AcCl (7.3 μL, 0.09 mmol, 0.98 eq), DIPEA (till basic pH), were stirred in a mixture of DMF: ACN (0.5 mL:0.5 mL) for 5 min over ice-bath and for 40 min at RT. The crude was injected to prep. HPLC, to provide the product as white solid powder.

¹H NMR (500 MHz, MeOD) δ ppm 1.88-1.96 (m, 4H), 2.79-2.81 (m, 4H), 5.83 (dd, J=9.17, 2.88 Hz, 1H), 6.40-6.51 (m. 2H), 7.45-7.50 (m, 2H), 7.60 (d, J=7.02 Hz, 1H), 8.20 (s, 1H). ¹³C-DEPTQ NMR (500 MHz, MeOD): δ ppm 22.42, 23.00, 24.01, 115.61, 119.90, 121.68, 123.76, 128.26, 130.71, 131.27, 132.31, 140.60, 144.92, 146.81, 166.28. HRMS: m/z, C₁₆H₁₈N₃O, required 268.1450 [M+H]⁺, found 268.1456 [M+H]⁺, 290.1276 [M+Na]⁺.

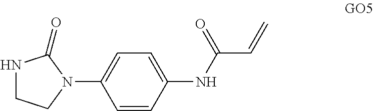 [SMILES: C=CC(=O)Nc1ccc(N2CCNC2=O)cc1]

Following general acrylation procedure, N-aryl precursor (available from a commercial source) (0.02 g, 0.11 mmol, 1 eq.), AcCl (10.0 μL, 0.12 mmol, 1.1 eq.), DIPEA (till basic pH), were stirred in a mixture of DMF: ACN (0.8 mL: 0.8 mL) for 30 min. over ice-bath and at RT for 2 hours. The product was obtained as white powder.

¹H NMR (500 MHz, Acetone-D6) δ ppm 3.55 (t, J=8 Hz, 2H), 3.94 (t, J=8 Hz, 2H), 5.68 (dd, J=10 Hz, 2 Hz, 1H), 6.33 (dd, J=17 Hz, 2 Hz, 1H), 6.46 (dd, J=17 Hz, 10 Hz, 1H), 7.59 (d, J=8 Hz, 2H), 7.69 (d, J=8 Hz, 2H). ¹³C NMR (125 MHz, Acetone-D6): δ ppm 36.92, 44.78, 117.29, 119.61, 119.70, 125.63, 132.02, 133.46, 137.12, 159.18. HRMS: m/z, C₁₂H₁₃N₃O₂Na, required 254.0905 [M+Na]⁺, found 254.0907 [M+Na]⁺, 485.1907 [2M+Na]⁺.

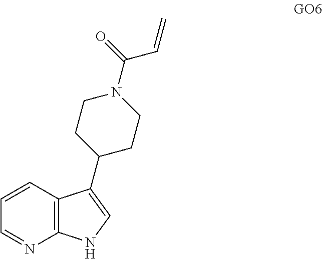 [SMILES: C=CC(=O)N1CCC(c2c[nH]c3ncccc23)CC1]

Following general acrylation procedure, N-aryl precursor (available from a commercial source) (0.02 g, 0.09 mmol, 1 eq.), AcCl (8.9 μL, 0.10 mmol, 1.1 eq.), DIPEA (till basic pH), were stirred in a mixture of DMF: ACN (0.5 mL: 0.5 mL) for 30 min. over ice-bath and at RT for 2 hours. The product was obtained as sticky oil.

¹H NMR (300 MHz, MeOD) δ ppm 1.82-1.65 (m, 2H), 2.19-2.14 (m, 2H), 3.00-2.90 (m, 1H), 3.42-3.25 (m, 1H), 4.30-4.26 (m, 1H), 4.76-4.72 (m, 1H) 5.77 (dd, J=11 Hz, 2 Hz, 1H), 6.23 (dd, J=17 Hz, 2 Hz, 1H), 6.84 (dd, J=17 Hz, 11 Hz, 1H), 7.55-7.50 (m, 2H), 8.39 (d, J=6 Hz, 1H), 8.74 (dd, J=8 Hz, 1.2 Hz, 1H). ESI-MS: m/z, C₅H₈N₃O, required 256.14 [M+H]⁺, found 256.12 [M+H]⁺, 278.24 [M+Na]⁺, 511.47 [2M+H]⁺, 533.45 [2M+Na]⁺, 788.67 [3M+Na]⁺. ESI-MS indicates also presence of impurities.

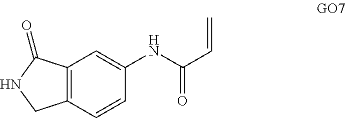

Following general acrylation procedure, N-aryl precursor (available from a commercial source) (0.02 g, 0.13 mmol, 1 eq.), AcCl (12.0 μL, 0.15 mmol, 1.1 eq.), DIPEA (till basic pH), were stirred in a mixture of DMF:ACN (0.8 mL: 0.7 mL) for 30 min. over ice-bath and at RT for 2 hours. The product was obtained as white powder.

¹H NMR (400 MHz, Acetone-D6) δ ppm 4.43 (s, 2H), 5.75 (dd, J=10 Hz, 2 Hz, 1H), 6.39 (dd, J=17 Hz, J=2 Hz, 1H), 6.49 (dd, J=17 Hz, 10 Hz, 1H), 7.53 (d, J=8 Hz, 1H), 7.94 (d, J=8 Hz, 1H), 8.21 (s, 1H). ¹³C NMR (100 MHz, Acetone-D6): δ ppm 44.57, 113.65, 122.67, 123.75, 126.43, 131.76, 133.60, 139.09, 163.28, 169.85. HRMS: m/z, C₁₁H₁₁N₂O₂, required 203.0820 [M+H]⁺, found 203.0814 [M+H]⁺, 225.0634 [M+Na]⁺, 427.1369 [2M+Na]⁺.

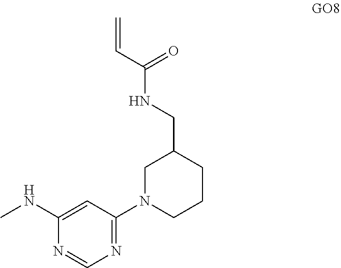 [SMILES: C=CC(=O)NCC1CCCN(c2cc(NC)ncn2)C1]

Following general acrylation procedure, N-aryl precursor (available from a commercial source) (0.02 g, 0.09 mmol, 1 eq.), AcCl (8.0 μL, 0.10 mmol, 1.1 eq.), DIPEA (till basic pH), were stirred in a mixture of DMF: ACN (0.7 mL: 0.7 mL) for 30 min. over ice-bath and at RT for 2 hours. The product was obtained as white powder.

¹H NMR (500 MHz, Acetone-D6) δ ppm 1.47-1.39 (m, 1H), 1.60-1.51 (m, 1H), 1.94-1.80 (m, 2H), 2.90 (s, 3H), 3.30-2.99 (m, 4H), 4.42-4.32 (m, 2H), 5.59 (dd, J=10 Hz, 2 Hz, 1H), 5.80 (s, 1H), 6.21 (dd, J=17 Hz, 2 Hz, 1H), 6.32 (dd, J=17 Hz, 10 Hz, 1H), 8.19 (s, 1H). ¹³C NMR (125 MHz, Acetone-D6): δ ppm 24.18, 27.35, 28.19, 36.59, 41.62, 45.39, 48.49, 77.55, 124.61, 131.70, 149.64, 156.30, 160.92, 165.08. HRMS: m/z, C₁₄H₂₂N₅O, required 276.1824 [M+H]⁺, found 276.1816 [M+H]⁺, 298.1631 [M+Na]⁺.

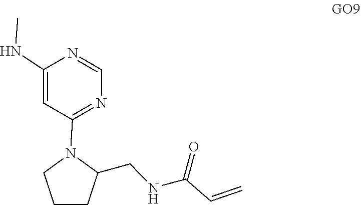 [SMILES: C=CC(=O)NCC1CCCN1c1cc(NC)ncn1]

Following general acrylation procedure, N-aryl precursor (available from a commercial source) (0.02 g, 0.09 mmol, 1 eq.), AcCl (8.6 μL, 0.10 mmol, 1.1 eq.), DIPEA (till basic pH), were stirred in a mixture of DMF: ACN (0.2 mL: 1.5 mL) for 30 min. over ice-bath and at RT for 2 hours. As the product is soluble in water, the crude was injected to prep. HPLC without extractions.

The product was obtained as colorless oil.

HRMS: m/z, C₁₃H₂₀N₅O, required 262.1668 [M+H]⁺, found 262.1667 [M+H]⁺.

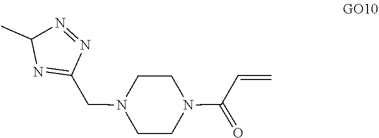 [SMILES: C=CC(=O)N1CCN(CC2=NC(C)N=N2)CC1]

Following general acrylation procedure, N-aryl precursor (available from a commercial source) (0.025 g, 0.11 mmol, 1 eq.), AcCl (10.3 μL, 0.13 mmol, 1.1 eq.), DIPEA (till basic pH), were stirred in a mixture of DMF: ACN (1.5 mL: 0.5 mL) for 30 min. over ice-bath and at RT overnight. As the product is soluble in water, the crude was injected to prep. HPLC without extractions. The product was obtained as colorless oil. NMR: degrades over time may contains impurities.

ESI-MS: m/z, C₁₁H₁₈N₅O, required 236.14 [M+H]⁺, found 236.24 [M+H]⁺, 258.23 [M+Na]⁺, 493.47 [2M+Na]⁺.

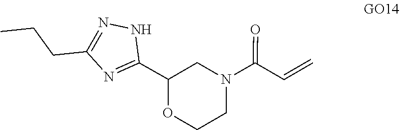 [SMILES: C=CC(=O)N1CCOC(c2nc(CCC)n[nH]2)C1]

Following general acrylation procedure, N-aryl precursor (available from a commercial source; 0.02 g, 0.10 mmol, 1 eq.), AcCl (9.1 μL, 0.11 mmol, 1.1 eq.), Et₃N (till basic pH), were stirred in ACN (2 mL) for 10 min. over ice-bath and at RT 2 hours. As the product is soluble in water, the crude was injected to prep. HPLC without extractions. The product was obtained as colorless oil.

HRMS: m/z, C₁₂H₁₈N₄O₂Na, required 273.1328 [M+Na]⁺, found 273.1323 [M+Na]⁺, 523.2736 [2M+Na]⁺.

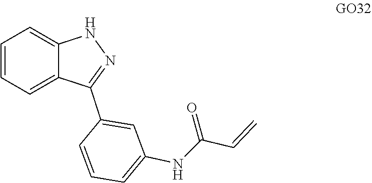 [SMILES: C=CC(=O)Nc1cccc(-c2n[nH]c3ccccc23)c1]

Following synthetic pathway B and general acrylation procedure, GO32 was prepared from commercially available 3-bromo-1H-indazole and (3-aminophenyl)boronic acid. Acrylation conditions: N-aryl precursor (0.34 gr, 1.13 mmol, 1 eq.), AcCl (90 μL, 1.13 mmol, 1 eq.), Et₃N (till basic pH), were stirred in a mixture of DMF: ACN (1 mL: 5 mL) for 30 min over ice-bath and for 30 min at RT. The crude was injected to prep. HPLC to provide the product as white solid powder.

¹H NMR (400 MHz, acetone-d6) δ ppm 5.77 (d, J=9.84 Hz, 1H), 6.43-6.59 (m, 2H), 7.24 (t, J=7.43 Hz, 1H), 7.42 (t, J=7.50 Hz, 1H), 7.49 (t, J=7.84 Hz, 1H), 7.65 (d, J=8.37 Hz, 1H), 7.81-7.85 (m, 3H), 8.18 (d, J=8.19 Hz, 1H), 8.60 (s, 1H) 9.70 (s, 1H). ¹³C-DEPTQ NMR (400 MHz, acetone-d6) δ ppm 110.75, 118.54, 119.08, 120.85, 121.02, 121.44, 122.65, 126.58, 126.78, 129.44, 132.09, 134.83, 139.80, 142.28, 144.07, 163.94. HRMS: m/z, C₁₆H₁₄N₃O, required 264.1137 [M+H]⁺, found 264.1146 [M+H]⁺, 286.0967 [M+Na]⁺.

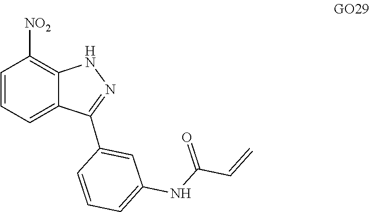 [SMILES: C=CC(=O)Nc1cccc(-c2n[nH]c3c([N+](=O)[O-])cccc23)c1]

Following synthetic pathway B and general acrylation procedure, GO29 was prepared from commercially available 3-bromo-7-nitro-1H-indazole and (3-aminophenyl)boronic acid. Acrylation conditions: N-aryl precursor (0.03 g, 0.085 mmol), AcCl (7.6 μL, 0.093 mmol, 1.1 eq.), DIPEA (till basic pH), were stirred in a mixture of DMF: ACN (0.5 mL: 0.5 mL) for 2 hours over-ice bath. The crude was injected to prep. HPLC, to provide the product as yellow solid powder.

¹H NMR (400 MHz, Acetone-D6) δ ppm 5.78 (dd, J=10 Hz, J=2 Hz, 1H), 6.68-6.35 (m, 2H), 7.57-7.52 (m, 2H), 7.81 (d, J=8 Hz, 1H), 7.84 (d, J=8 Hz, 1H), 8.47 (d, J=8 Hz, 1H), 8.58 (s, 1H), 8.66 (d, J=8 Hz, 1H). ¹³C NMR (100 MHz, Acetone-D6): δ ppm 118.46, 119.42, 120.91, 122.42, 122.65, 123.55, 124.95, 125.99, 126.49, 129.46, 129.58, 131.85, 133.12, 134.09, 139.86, 163.37. HRMS: m/z, C₁₆H₁₂N₄O₃Na, required 331.0807 [M+Na]⁺, found 331.0800 [M+Na]⁺, 639.1739 [2M+Na]⁺.

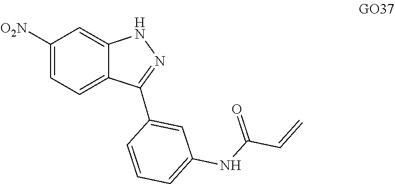 [SMILES: C=CC(=O)Nc1cccc(-c2n[nH]c3cc([N+](=O)[O-])ccc23)c1]

Following synthetic pathway B and general acrylation procedure, GO37 was prepared from commercially available 3-bromo-6-nitro-1H-indazole and (3-aminophenyl)boronic acid. Acrylation conditions: N-aryl precursor (0.04 g, 0.11 mmol), AcCl (8.5 μL, 0.10 mmol, 0.95 eq.), Et3N (till basic pH), were stirred in a mixture of DMF: ACN (1 mL: 1 mL) for 30 min over ice-bath and for 60 min at RT. The crude was injected to prep. HPLC, to provide the product as yellow solid powder.

¹H NMR (500 MHz, Acetone-D6) δ ppm 5.77 (dd, J=10 Hz, 2 Hz, 1H), 6.42 (dd, J=17 Hz, 2 Hz, 1H), 6.54 (dd, J=17 Hz, 10 Hz, 1H), 7.52 (t, J=8 Hz, 1H), 7.82-7.80 (m, 2H), 8.11 (dd, J=9 Hz, 1.6 Hz, 1H), 8.38 (d, J=9 Hz, 1H) 8.59 (s, 1H), 8.63 (d, J=1.6 Hz, 1H). ¹³C NMR (125 MHz, Acetone-D6): δ ppm 107.35, 115.49, 118.22, 119.26, 121.92, 122.31, 123.62, 126.44, 129.40, 131.93, 133.85, 139.95, 141.01, 146.61, 148.10, 163.49. HRMS: m/z, C₁₆H₁₁N₄O₃, required 307.0831 [M−H]⁻, found 307.0831 [M−H]⁻.

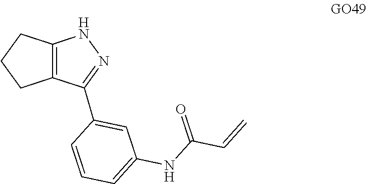 [SMILES: C=CC(=O)Nc1cccc(-c2n[nH]c3c2CCC3)c1]

Following general acrylation procedure, N-aryl precursor (available from a commercial source) (0.02 g, 0.10 mmol, 1 eq.), AcCl (7.8 μL, 0.098 mmol, 0.98 eq.), DIPEA (till basic pH), were stirred in a mixture of DMF: ACN (0.4 mL: 0.4 mL) for 10 min. over ice-bath and at RT for 30 min. The crude was injected to prep. HPLC, to provide the product as white solid powder.

HRMS: m/z, C₁₅H₁₆N₃O, required 254.1293 [M+H]⁺, found 254.1288 [M+H]⁺, 276.1113 [M+Na]⁺, 507.2504 [2M+H]⁺, 529.2320 [2M+Na]⁺.

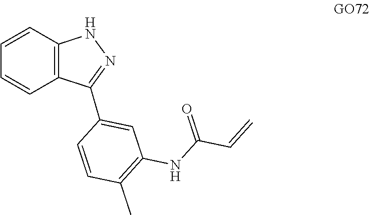 [SMILES: C=CC(=O)Nc1cc(-c2n[nH]c3ccccc23)ccc1C]

Following synthetic pathway B and general acrylation procedure, GO72 was prepared from commercially available 3-bromo-1H-indazole and (3-amino-4-methylphenyl)boronic acid. Acrylation conditions: N-aryl precursor (0.031 g, 0.092 mmol), AcCl (63 μL, 0.078 mmol, 0.86 eq.), DIPEA (till basic pH), were stiffed in a mixture of DMF: ACN (1 mL: 1 mL) for 5 min over ice-bath and for 30 min at RT. The crude was injected to prep. HPLC, to provide the product as white solid powder.

¹H NMR (300 MHz, MeOD) ppm δ 2.35 (s, 3H), 5.83 (dd, J=10.10, 1.43 Hz, 1H), 6.39-6.63 (m, 2H), 7.20 (t, J=8.3 Hz, 1H), 7.39-7.45 (m, 2H), 7.56 (d, J=8.30 Hz, 1H), 7.75 (d, J=7.91 Hz, 1H), 8.05-8.10 (m, 2H). ¹³C NMR (300 MHz, MeOD) ppm 6 18.01, 111.47, 121.77, 122.01, 122.41, 125.63, 126.21, 127.89, 128.08, 132.18, 132.23, 133.15, 133.65, 137.18, 143.33, 145.49, 166.84. HRMS: m/z, C₁₇H₆N₃O, required 278.1293 [M+H]⁺, found 278.1282 [M+H]⁺, 300.1105 [M+Na]⁺, 555.2492 [2M+H]⁺, 577.2313 [2M+Na]⁺.

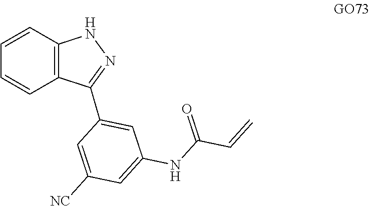 [SMILES: C=CC(=O)Nc1cc(C#N)cc(-c2n[nH]c3ccccc23)c1]

Following synthetic pathway B and general acrylation procedure, GO73 was prepared from commercially available 3-bromo-1H-indazole and (3-amino-5-cyanophenyl)boronic acid. Acrylation conditions: N-aryl precursor (0.026 g, 0.075 mmol), AcCl (7 μL, 0.086 mmol, 0.88 eq.), DIPEA (till basic pH), were stirred in a mixture of DMF: ACN (1 mL: 1 mL) for 5 min over ice-bath and for 1 hour at RT. The crude was injected to prep. HPLC, to provide the product as white solid powder.

¹H NMR (500 MHz, Acetone-D6) δ ppm 5.83 (dd, J=10 Hz, 2 Hz, 1H), 6.45 (dd, J=17 Hz, 2 Hz, 1H), 6.53 (dd, J=17 Hz, 10 Hz, 1H), 7.32 (t, J=8 Hz, 1H), 7.48 (t, J=8 Hz, 1H), 7.70 (d, J-8 Hz, 1H), 8.13 (s, 1H), 8.23 (d, J=8 Hz, 1H), 8.26 (s, 1H), 8.75 (s, 1H). ¹³C NMR (125 MHz, Acetone-D6): δ ppm 110.71, 113.19, 118.36, 120.43, 120.60, 120.84, 121.53, 121.72, 124.81, 126.52, 127.40, 131.37, 136.28, 140.58, 141.74, 142.04, 163.75. HRMS: m/z, C₁₇H₁₁N₄O, required 287.0933 [M−H]⁻, found 287.0934 [M−H]⁻.

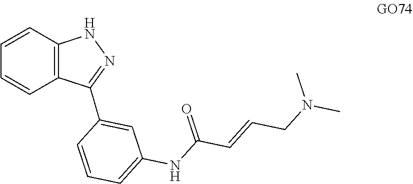 [SMILES: CN(C)C/C=C/C(=O)Nc1cccc(-c2n[nH]c3ccccc23)c1]

Following synthetic pathway B, GO74 was prepared from commercially available 3-bromo-1H-indazole and (3-aminophenyl)boronic acid. After Boc acidolysis, the N-aryl precursor was coupled to 4-(Dimethylamino)-2-butenoic acid hydrochloride, using HATU coupling reagent. Coupling procedure: 4-(Dimethylamino)-2-butenoic acid (0.012 g, 0.07 mmol, 0.92 eq.) was dissolved in mixture of DMF: ACN (0.5 mL: 2 mL), DIPEA was added (till basic pH). HATU (0.030 g, 0.08 mmol, 0.98 eq.) was added to the reaction mixture, after stirring the mixture for 5 min. Following addition of N-aryl precursor (0.025 gr, 0.077 mmol, 1 eq.), the pH was adjusted to basic (pH 8-9) as required. The reaction was stirred at RT for 48 hours. After completing the reaction, the solvent was removed in vacuo and the residue was suspended in EtOAc, and washed with saturated NaHCO₃ solution. The organic extract was dried over Na₂SO₄ and evaporated in vacuo to yield the crude product, which was injected to prep. HPLC, to provide the product as yellowish powder.

¹H NMR (300 MHz, MeOD) δ ppm 2.93 (s, 6H), 4.05 (dd, J=7.19, 0.85 Hz, 2H), 6.59 (d, J=15.20 Hz, 1H), 6.88 (td, J=14.64, 7.22, 7.22 Hz, 1H), 7.23 (t, J=9.01, 1H), 7.41-7.59 (m, 3H), 7.65-7.75 (m, 2H), 8.06 (d, J=9.00 Hz, 1H), 8.36 (s, 1H). ¹³C NMR (300 MHz, MeOD) δ ppm 43.20, 58.78, 111.49, 120.25, 120.64, 121.67, 121.82, 122.43, 124.56, 127.85, 130.45, 132.11, 134.25, 135.62, 139.97, 143.28, 145.40, 164.32. HRMS: m/z, C₁₉H₂₁N₄O, required 321.1715 [M+H]⁺, found 321.1709 [M+H]⁺.

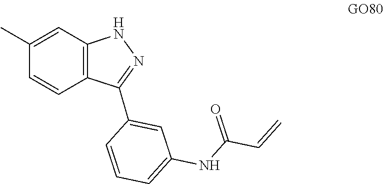 [SMILES: C=CC(=O)Nc1cccc(-c2n[nH]c3cc(C)ccc23)c1]

Following synthetic pathway B and general acrylation procedure, GO80 was prepared from commercially available 3-iodo-6-methyl-1H-indazole and (3-aminophenyl)boronic acid. Acrylation conditions: N-aryl precursor (0.62 g, 1.86 mmol), AcCl (148 μL, 1.86 mmol, 1 eq.), Et₃N (till basic pH), were stirred in a mixture of DMF: ACN (4 mL: 6 mL) for 30 min over ice-bath and for 30 min at RT. The crude was injected to prep. HPLC, to provide the product as white solid powder.

¹H NMR (400 MHz, acetone-d6) δ ppm 2.46 (s, 3H), 5.73 (dd, J=10 Hz, 2 Hz, 1H), 6.42 (dd, J=17 Hz, 2 Hz, 1H), 6.54 (dd, J=17 Hz, 10 Hz, 1H), 7.08 (d, J=8.4 Hz, 1H), 7.41 (s, 1H), 7.47 (t, J=8 Hz, 1H), 7.79-7.82 (m, 2H), 8.02 (d, J=8 Hz, 1H), 8.53 (s, 1H), 9.57 (s, 1H). ¹³C-DEPTQ NMR (400 MHz, acetone-d6) δ ppm 21.09, 109.98, 118.24, 118.78, 119.19, 120.61, 122.37, 123.50, 126.57, 129.30, 132.22, 135.17, 136.48, 139.87, 142.98, 143.84, 163.74. HRMS: m/z, C₁₇H₁₆N₃O, required 278.1293 [M+H]⁺, found 278.1294 [M+H]⁺, 300.1114 [M+Na]⁺.

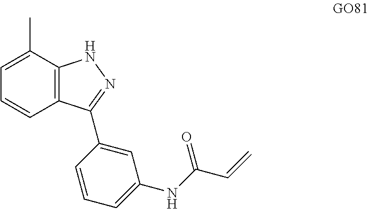 [SMILES: C=CC(=O)Nc1cccc(-c2n[nH]c3c(C)cccc23)c1]

Following synthetic pathway B and general acrylation procedure, GO81 was prepared from commercially available 3-bromo-7-methyl-1H-indazole and (3-aminophenyl)boronic acid. Acrylation conditions: N-aryl precursor (0.072 g, 0.21 mmol), AcCl (16 μL, 0.20 mmol, 0.95 eq.), DIPEA (till basic pH), were stiffed in a mixture of DMF: ACN (0.5 mL: 5 mL) for 5 min over ice-bath and for 1 hour at RT. The crude was injected to prep. HPLC, to provide the product as white solid powder.

¹H NMR (300 MHz, MeOD+CDCl₃) δ ppm 2.53 (s, 3H), 5.71 (dd, J=8.37, 3.25 Hz, 1H), 6.26-6.44 (m, 2H), 7.03-7.18 (m, 2H), 7.42 (t, J=7.92, 1H), 7.64 (d, J=7.74 Hz, 1H), 7.81-7.85 (m, 2H), 7.98 (s, 1H). ¹³C NMR (300 MHz, MeOD+CDCl₃) δ ppm 16.50, 118.48, 118.78, 119.79, 120.25, 120.53, 121.74, 123.31, 126.77, 127.37, 129.34, 131.14, 133.97, 136.18, 138.77, 141.80, 144.94, 165.81. HRMS: m/z, C₁₇H₁₆N₃O, required 278.1293 [M+H]⁺, found 278.1293 [M+H]⁺, 300.1113 [M+Na]⁺, 555.2512 [2M+H]⁺, 577.2329 [2M+Na]⁺.

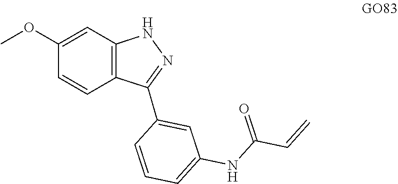 [SMILES: C=CC(=O)Nc1cccc(-c2n[nH]c3cc(OC)ccc23)c1]

Following synthetic pathway B and general acrylation procedure, GO83 was prepared from commercially available 3-iodo-6-methoxy-1H-indazole and (3-aminophenyl)boronic acid. Acrylation conditions: N-aryl precursor (0.073 g, 0.20 mmol), AcCl (15 μL, 0.19 mmol, 0.92 eq.), DIPEA (till basic pH), were stirred in a mixture of DMF: ACN (0.5 mL: 2 mL) for 5 min over ice-bath and for 30 min at RT. The crude was injected to prep. HPLC, to provide the product as white solid powder.

¹H NMR (300 MHz, MeOD) δ ppm 3.89 (s, 3H), 5.81 (dd, J=9.41, 2.21 Hz, 1H), 6.32-6.59 (m, 2H), 6.89 (dd, J=9.30, 3.10, 1H), 6.97 (s, 1H), 7.48 (t, J=9.20, 1H), 7.67-7.69 (m, 2H), 7.93 (dd, J=8.92, 1.87 Hz, 1H), 8.32 (s, 1H). ¹³C NMR (300 MHz, MeOD) δ ppm 55.96, 91.92, 115.26, 116.39, 120.28, 120.89, 122.86, 124.26, 128.03, 130.47, 132.52, 135.06, 140.33, 144.68, 145.44, 161.55, 166.34. HRMS: m/z, C₁₇H₆N₃O₂, required 294.1242 [M+H]⁺, found 294.1242 [M+H]⁺. 316.1059 [M+Na]⁺. 587.2411 [2M+H]⁺. 609.2228 [2M+Na]⁺.

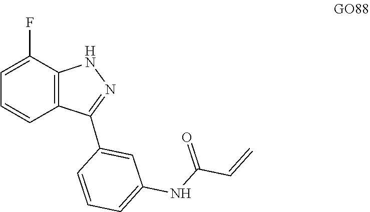 [SMILES: C=CC(=O)Nc1cccc(-c2n[nH]c3c(F)cccc23)c1]

Following synthetic pathway B and general acrylation procedure, GO88 was prepared from commercially available 3-bromo-7-fluoro-1H-indazole and (3-aminophenyl)boronic acid. Acrylation conditions: N-aryl precursor (0.05 g, 0.14 mmol), AcCl (11.9 μL, 0.15 mmol, 1 eq.), Et₃N (till basic pH), were stirred in a mixture of DMF: ACN (0.5 mL: 2 mL) for 30 min over ice-bath and for 60 min at RT. HPLC purification provided the product as white solid powder.

¹H NMR (400 MHz, Acetone-D6) δ ppm 5.76 (dd, J=10 Hz, 2 Hz, 1H), 6.41 (dd, J=17 Hz, 2 Hz, 1H), 6.54 (dd, J=17 Hz, 10 Hz, 1H), 7.27-7.19 (m, 2H), 7.49 (t, J=8 Hz, 1H), 7.80 (dd, J=8 Hz, 1.4 Hz, 2H), 7.99 (dd, J=8 Hz, 1 Hz, 1H) 8.57 (s, 1H). ¹³C NMR (100 MHz, Acetone-D6): 6 ppm 110.22 (d, J_{CF}=16 Hz), 116.89 (d, J_{CF}=4 Hz), 118.10, 118.86, 121.70 (d, J_{CF}=5.5 Hz), 122.26, 124.63 (d, J_{CF}=4 Hz), 126.35, 129.24, 131.51 (d, J_{CF}=16 Hz), 131.93, 134.07, 139.74, 144.74, 148.3 (d, J_{CF}=250 Hz), 163.34. HRMS: m/z, C₁₆H₁₂FN₃ONa, required 304.0862 [M+Na]⁺, found 304.0855 [M+Na]⁺, 563.2024 [2M+H]⁺, 585.1821 [2M+Na]⁺.

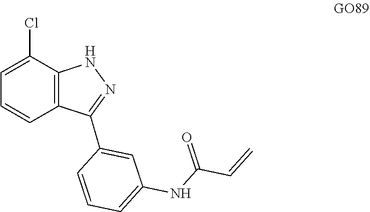 [SMILES: C=CC(=O)Nc1cccc(-c2n[nH]c3c(Cl)cccc23)c1]

Following synthetic pathway B and general acrylation procedure, GO89 was prepared from commercially available 7-chloro-3-iodo-1H-indazole and (3-aminophenyl)boronic acid. Acrylation conditions: N-aryl precursor (0.053 g, 0.15 mmol), AcCl (11.9 μL, 0.15 mmol, 1 eq.), Et3N (till basic pH), were stirred in a mixture of DMF: ACN (0.5 mL: 2 mL) for 30 min over ice-bath and for 60 min at RT. HPLC purification provided the product as white solid powder.

¹H NMR (400 MHz, Acetone-D6) δ ppm 5.76 (dd, J=10 Hz, 2 Hz, 1H), 6.41 (dd, J=17 Hz, 2 Hz, 1H), 6.54 (dd, J=17 Hz, 10 Hz, 1H), 7.29 (t, J=8 Hz, 1H), 7.52-7.47 (m, 2H), 7.80-7.78 (m, 2H), 8.15 (d, J=8 Hz, 1H) 8.56 (s, 1H). ¹³C NMR (100 MHz, Acetone-D6): δ ppm 115.64, 117.00, 118.09, 118.90, 119.85, 122.13, 122.26, 122.55, 125.64, 126.33, 129.26, 129.68, 131.94, 134.09, 139.77, 163.32. HRMS: m/z, C₁₆H₁₂ClN₃ONa, required 320.0567 [M+Na]⁺, found 320.0562 [M+Na]⁺, 617.1231 [2M+Na]⁺.

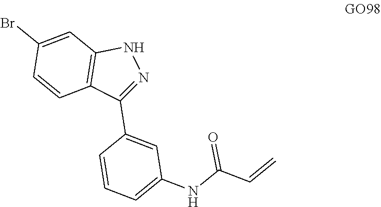 [SMILES: C=CC(=O)Nc1cccc(-c2n[nH]c3cc(Br)ccc23)c1]

Following synthetic pathway B and general acrylation procedure, GO98 was prepared from commercially available 6-bromo-3-iodo-1H-indazole and (3-aminophenyl)boronic acid. Acrylation conditions: N-aryl precursor (0.06 g, 0.15 mmol), AcCl (12 μL, 0.15 mmol, 1 eq.), Et₃N (till basic pH), were stirred in a mixture of DMF: ACN (0.4 mL:1 mL) for 30 min over ice-bath and for 30 min at RT. The crude was injected to prep. HPLC, to provide the product as white solid powder.

¹H NMR (400 MHz, Acetone-D6) δ ppm 5.77 (dd, J=10 Hz, 2 Hz, 1H), 6.41 (dd, J=17 Hz, 2 Hz, 1H), 6.53 (dd, J=17 Hz, 10 Hz, 1H), 7.40 (dd, J=8.7 Hz, 1.5 Hz, 1H), 7.49 (t, J=8 Hz, 1H), 7.80-7.77 (m, 2H), 7.88 (d, J=1.5 Hz, 1H) 8.11 (d, J=8.7 Hz, 1H), 8.54 (s, 1H). ¹³C NMR (100 MHz, Acetone-D6): δ ppm 113.33, 118.00, 118.81, 119.67, 119.99, 122.20, 122.45, 124.37, 126.36, 129.25, 131.90, 134.11, 139.71, 142.78, 144.22, 163.29. HRMS: m/z, C₁₆H₁₁BrN₃O, required 340.0085 [M−H]⁻, found 340.0088 [M−H]⁻.

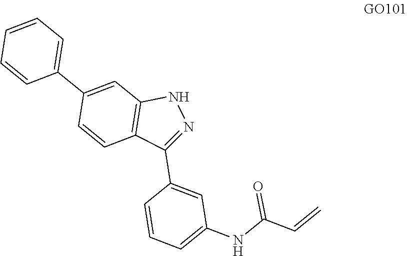 [SMILES: C=CC(=O)Nc1cccc(-c2n[nH]c3cc(-c4ccccc4)ccc23)c1]

Following synthetic pathway B and general acrylation procedure, GO101 was prepared from commercially available 6-bromo-3-iodo-1H-indazole and (3-aminophenyl) boronic acid. Prior to Boc acidolysis the bromo group was converted to phenyl by additional Suzuki cross coupling reaction. Suzuki reaction with phenylboronic acid using the same conditions as described in general procedure (vide supra) afforded the desired phenyl in position 6. Acrylation conditions: N-aryl precursor (0.05 g, 0.12 mmol), AcCl (11 μL, 0.14 mmol, 1.1 eq.), Et₃N (till basic pH), were stirred in a mixture of DMF: ACN (0.6 mL: 2 mL) for 30 min over ice-bath and for 60 min at RT. The crude was injected to prep. HPLC, to provide the product as off-white solid powder.

¹H NMR (500 MHz, Acetone-D6) δ ppm 5.76 (dd, J=10 Hz, 2 Hz, 1H), 6.42 (dd, J=17 Hz, 2 Hz, 1H), 6.55 (dd, J=17 Hz, 10 Hz, 1H), 7.41 (t, J=7.5 Hz, 1H), 7.54-7.48 (m, 3H), 7.58 (d, J=8.5 Hz, 1H), 7.78 (d, J=7.5 Hz, 2H), 7.81 (d, J=8 Hz, 1H), 7.85 (d, J=8, 1H) 7.88 (s, 1H), 8.26 (d, J=8.5 Hz, 1H), 8.61 (s, 1H). ¹³C NMR (125 MHz, Acetone-D6): δ ppm 108.32, 117.95, 118.55, 120.00, 121.08, 121.24, 122.12, 126.26, 127.34, 127.45, 128.90, 129.16, 131.99, 134.76, 139.39, 139.71, 141.15, 142.80, 143.77, 163.40. HRMS: m/z, C₂₂H₁₇N₃ONa, required 362.1269 [M+Na]⁺, found 362.1261 [M+Na]⁺, 701.2646 [2M+Na]⁺.

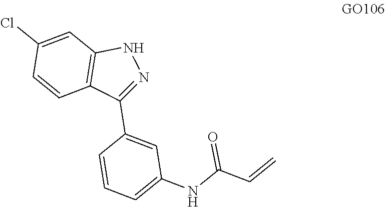 [SMILES: C=CC(=O)Nc1cccc(-c2n[nH]c3cc(Cl)ccc23)c1]

Following synthetic pathway B and general acrylation procedure, GO106 was prepared from commercially available 6-chloro-3-iodo-1H-indazole and (3-aminophenyl)boronic acid. Acrylation conditions: N-aryl precursor (0.065 g, 0.82 mmol), AcCl (17 μL, 0.22 mmol, 1.2 eq.), Et₃N (till basic pH), were stirred in a mixture of DMF: ACN (1 mL: 2 mL) for 30 min over ice-bath and for 30 min at RT. The crude was injected to prep. HPLC, to provide the product as white solid powder.

¹H NMR (500 MHz, Acetone-D6) δ ppm 5.76 (dd, J=10 Hz, 2 Hz, 1H), 6.41 (dd, J=17 Hz, 2 Hz, 1H), 6.52 (dd, J=17 Hz, 10 Hz, 1H), 7.27 (dd, J=9 Hz, 1.5 Hz, 1H), 7.49 (t, J=8 Hz, 1H), 7.71 (d, J=1.5 Hz, 1H), 7.80-7.77 (m, 2H), 8.16 (d, J=9 Hz 1H), 8.54 (s, 1H). ¹³C NMR (125 MHz, Acetone-D6): δ ppm 110.13, 117.99, 118.79, 119.44, 121.82, 122.19, 122.24, 126.36, 129.24, 130.28, 131.90, 134.14, 139.71, 142.77, 144.24, 163.29. HRMS: m/z, C₁₆H₁₃ClN₃O, required 298.0747 [M+H]⁺, found 298.0744 [M+H]⁺, 320.0565 [M+Na]⁺, 617.1245 [2M+Na]⁺.

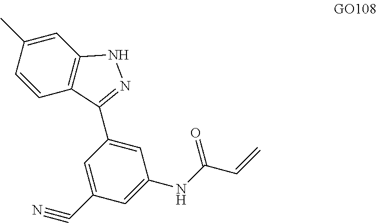 [SMILES: C=CC(=O)Nc1cc(C#N)cc(-c2n[nH]c3cc(C)ccc23)c1]

Following synthetic pathway B and general acrylation procedure, GO108 was prepared from commercially available 3-iodo-6-methyl-1H-indazole and (3-amino-5-cyanophenyl)boronic acid. Acrylation conditions: N-aryl precursor (0.05 g, 0.15 mmol), AcCl (11.8 μL, 0.15 mmol, 1 eq.), Et₃N (till basic pH), were stirred in a mixture of DMF: ACN (1 mL: 2 mL) for 30 min over ice-bath and for 1.5 at RT. The crude was injected to prep. HPLC, to provide the product as white solid powder.

¹H NMR (500 MHz, Acetone-D6) δ ppm 2.51 (s, 3H), 5.83 (dd, J=10 Hz, 2 Hz, 1H), 6.47 (dd, J=17 Hz, 2 Hz, 1H), 6.52 (dd, J=17 Hz, 10 Hz, 1H), 7.16 (d, J=8.4 Hz, 1H), 7.47 (s, 1H), 8.09 (d, J=8.4 Hz, 1H), 8.12 (s, 1H), 8.25 (s, 1H), 8.72 (s, 1H). ¹³C NMR (125 MHz, Acetone-D6): δ ppm 20.86, 109.97, 113.15, 118.38, 118.65, 120.04, 120.74, 121.39, 123.92, 124.70, 127.38, 131.37, 136.42, 136.73, 140.55, 141.49, 142.72, 163.73. HRMS: m/z, C₁₈H₁₄N₄ONa, required 325.1065 [M+Na]⁺, found 325.1066 [M+Na]⁺, 627.2239 [2M+Na]⁺.

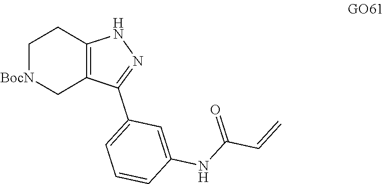 [SMILES: C=CC(=O)Nc1cccc(-c2n[nH]c3c2CN(C(=O)OC(C)(C)C)CC3)c1]

Following synthetic pathway A, GO61 was prepared from commercially available 4 N-Boc-4-piperidone and 3-aminobenzoic acid. Acrylation conditions: N-aryl precursor (0.05 g, 0.16 mmol), AcCl (11.4 μL, 0.14 mmol, 0.9 eq.), DIPEA (till basic pH), were stirred in a mixture of DMF: ACN (0.5 mL: 2 mL) for 10 min over ice-bath and for 30 min at RT, the reaction was quenched by addition of 1 mL of water. The crude was injected to prep. HPLC, to provide the product as white solid powder.

HRMS: m/z, C₂₀H₂₄N₄O₃Na, required 391.1746 [M+Na]⁺, found 391.1735 [M+Na]⁺, 737.3796 [2M+H], 759.3611 [2M+Na]⁺.

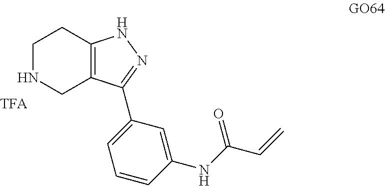 [SMILES: C=CC(=O)Nc1cccc(-c2n[nH]c3c2CNCC3)c1]

GO64 was prepared by Boc group acidosis from available GO61. Acidolysis conditions: GO61 (0.06 g, 0.16 mmol, 1 eq.), DCM (2 mL), TFA (2 mL), a drop of Triisopropylsilane. The reaction mixture was stirred at RT for 30 min. The solvent was removed in vacuo to yield the product.

¹H NMR (300 MHz, MeO)D) δ ppm 1.30 (s, 1H), 3.11 (t, J=6.20 Hz, 2H), 3.60 (t, J=6.20 Hz, 2H), 4.48 (s, 2H), 5.81 (dd, J=9.00, 3.00 Hz, 1H), 6.35-6.52 (m, 2H), 7.35-7.50 (m, 4H), 8.08 (s, 1H). ¹³C NMR (300 MHz, MeOD) δ ppm 20.81, 42.30, 42.81, 106.70, 119.41, 121.12, 123.29, 127.77, 128.23, 130.82, 132.40, 140.61, 141.91, 144.26, 166.39. HRMS: m/z, C₁₅H₁₇N₄O, required 269.1402 [M+H]⁺, found 269.1400 [M+H]⁺, 537.2713 [2M+H]⁺.

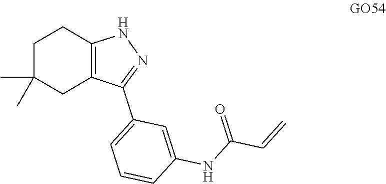 [SMILES: C=CC(=O)Nc1cccc(-c2n[nH]c3c2CC(C)(C)CC3)c1]

Following synthetic pathway A, GO54 was prepared from commercially available 4,4-dimethylcyclohexan-1-one and 3-aminobenzoic acid. Acrylation conditions: N-aryl precursor (0.04 g, 0.16 mmol), AcCl (11 μL, 0.13 mmol, 0.8 eq.), DIPEA (till basic pH), were stirred in a mixture of DMF: ACN (2 mL: 1 mL) for 10 min over ice-bath and for 20 min at RT, the reaction was quenched by addition of 1 mL of water. The crude was injected to prep. HPLC, to provide the product as white solid powder.

¹H NMR (300 MHz, MeOD δ ppm 1.07 (s, 6H), 1.72 (t, J=6.60 Hz, 2H), 2.56 (s, 2H), 2.79 (t, J=6.60 Hz, 2H), 5.82 (dd, J=8.85, 2.70 Hz, 1H), 6.42-6.47 (m, 2H), 7.39-7.50 (m, 2H), 7.63 (d, J=7.80, 1H), 8.10 (s, 1H). ¹³C NMR (300 MHz, MeOD) δ ppm 19.59, 28.03, 31.22, 35.79, 36.16, 115.21, 119.83, 121.51, 123.82, 128.19, 130.62, 131.65, 132.32, 140.60, 144.62, 145.67, 166.27. ¹H NMR (400 MHz, Acetone-D6) δ ppm 1.04 (s, 6H), 1.64 (t, J=6.5 Hz, 2H), 2.55 (s, 2H), 2.70 (t, J=6.5 Hz, 2H), 5.72 (dd, J=10 Hz, 2 Hz, 1H), 6.37 (dd, J=17 Hz, 2 Hz, 1H), 6.49 (dd, J=17 Hz, 10 Hz, 1H), 7.37 (t, J=8 Hz, 1H), 7.46 (d, J=8 Hz, 1H), 7.75 (d, J=8, 1H), 8.11 (s, 1H). ¹³C NMR (100 MHz, Acetone-D6): δ ppm 18.80, 27.39, 30.25, 35.28, 36.04, 112.13, 117.27, 117.95, 121.57, 126.15, 128.81, 131.96, 134.39, 139.44, 141.50, 144.39, 163.16. HRMS: m/z, C₁₈H₂₂N₃O, required 296.1763 [M+H]⁺, found 296.1762 [M+H]⁺, 318.1581 [M+Na]⁺, 591.3445 [2M+H]⁺, 613.3264 [2M+Na]⁺.

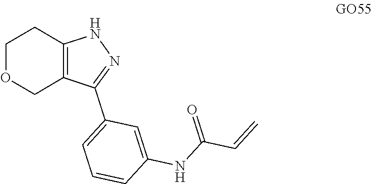 [SMILES: C=CC(=O)Nc1cccc(-c2n[nH]c3c2COCC3)c1]

Following synthetic pathway A, GO55 was prepared from commercially available Tetrahydro-4H-pyran-4-one and 3-aminobenzoic acid. Acrylation conditions: N-aryl precursor (0.025 g, 0.11 mmol), AcCl (7.4 μL, 0.09 mmol, 0.8 eq.), DIPEA (till basic pH), were stirred in a mixture of DMF: ACN (0.6 mL: 2 mL) for 10 min over ice-bath and for 30 min at RT, the reaction was quenched by addition of 1 mL of water. The crude was injected to prep. HPLC, to provide the product as white solid powder.

¹H NMR (400 MHz, Acetone-D6) δ ppm 2.80 (t, J=5.5 Hz, 2H), 3.93 (t, J=5.5 Hz, 2H), 4.90 (s, 2H), 5.74 (dd, J=10 Hz, 2 Hz, 1H), 6.38 (dd, J=17 Hz, 2 Hz, 1H), 6.50 (dd, J=17 Hz, 10 Hz, 1H), 7.39-7.37 (m, 2H), 7.70-7.67 (m, 1H), 8.05 (s, 1H), 7.88 (d, J=1.5 Hz, 1H) 8.11 (d, J=8.7 Hz, 1H), 8.54 (s, 1H). ¹³C NMR (100 MHz, Acetone-D6): δ ppm 23.14, 63.88, 64.15, 110.91, 111.39, 116.81, 118.06, 120.88, 126.27, 129.12, 131.90, 139.70, 140.19, 140.42, 163.21. HRMS: m/z, C₁₅H₁₆N₃O₂, required 270.1242 [M+H]⁺, found 270.1244 [M+H]⁺, 292.1063 [M+Na]⁺, 539.2415 [2M+H]⁺, 561.2233 [2M+Na]⁺.

SAR-Based Covalent Inhibitor Design:

Based on SAR analysis and drug-utility assays, resented hereinbelow, an additional series of compounds, suitable for use as covalent inhibitors of MKK7, according to embodiments of the present invention, were prepared and listed in Table 3 below, wherein EC50 in HEK293 and 3T3 WT cells are ICW assessment of pJNK inhibition.

TABLE 3

| | | | EC₅₀ HEK293 | EC₅₀ 3T3 WT |
|---|---|---|---|---|
| Compound | Structure | cLogP | (μM)^{a} | (μM)^{a} |
| PCM475 | 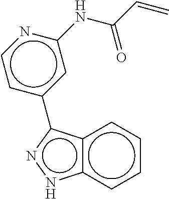 | 2.7 | 0.9 | 2.8 0.3 0.3 |
| PCM476 | 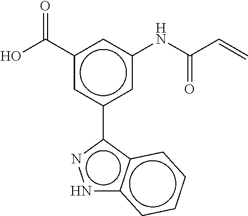 | −0.55 | | >10 |
| PCM487 | 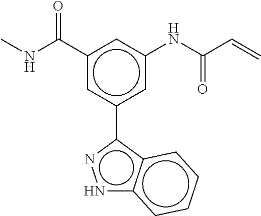 | 2.4 | | 3.0 6.3 >10 |
| PCM531 | 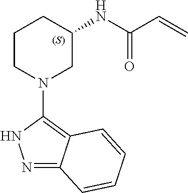 | 2.24 | >10 | |
| PCM532 | 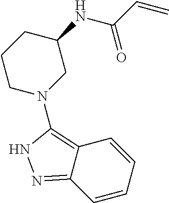 [SMILES: C=CC(=O)N[C@@H]1CCCN(c2[nH]nc3ccccc23)C1] | 2.24 | >10 | |
| PCM542 | 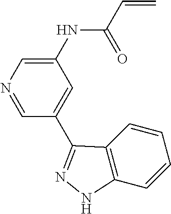 [SMILES: C=CC(=O)Nc1cncc(-c2n[nH]c3ccccc23)c1] | 2.11 | | 6.8 3.0 |
| PCM548 | 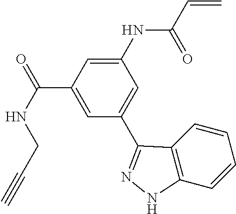 [SMILES: C#CCNC(=O)c1cc(NC(=O)C=C)cc(-c2n[nH]c3ccccc23)c1] | 2.63 | | 4.3 6.3 |
| PCM549 | 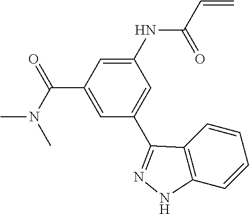 [SMILES: C=CC(=O)Nc1cc(C(=O)N(C)C)cc(-c2n[nH]c3ccccc23)c1] | 2.62 | | >10 |

### Example 3

### Methods

In Cell Western (ICW):

HEK293, Beas2B and 3T3 cells were plated 24 hours before procedure on 384 well clear bottom black flat plates (Corning) pre-coated with Fibronectin (Sigma) diluted in PBS to a concentration of 5 μg/ml 45 minutes prior to cells introduction. Cells were plated using Multidrop™ Combi reagent dispenser (Thermo Fisher Scientific). Compound, reagents and antibody dispensing as well as washes were performed with a CyBio liquid handler (Analytik). Cells were pre-incubated with a small molecule or up to 1% DMSO for 2 h before fixation. HEK293, 3T3 or Beas2B cells were treated with 0.6M/0.2M/0.2M D-Sorbitol/PBS (Sigma) for 20/40/40 min. respectively, before fixation. Media was removed and wells were immediately fixed with 150 μl per well of 3.7% formaldehyde/PBS fixing solution for 20 min. Formaldehyde was then aspired and cells were permeabilized with 150 μl of 0.5% Triton x-100/PBS solution for 10 min. After aspiration cells were washed with ice-cold methanol and stored at −20° C., for 10 min. Methanol was aspired and cells were blocked with 150 μl blocking buffer (LICOR) for 90 min. Primary antibody Phospho-SAPK/JNK T183/Y185 (CST; 9251S) was diluted 1:1000 in blocking buffer and was applied 50 μl per well, except control wells to which only blocking buffer was applied, O.N. (16 h) at 4° C. with mild shaking. Primary antibody was aspired, wells were washed with 150 μl 0.1% Tween20/PBS (Sigma) washing solution three times for 7 min. IRDye® 800CW Goat anti-Rabbit IgG Secondary antibody (LICOR) was then diluted 1:1200 in blocking buffer with 50 μl applied per well on control wells only (no CellTag control), then CellTag700 (1:2000) was added to the solution and 50 μl were added to all experiment wells, for 1 h at room temperature. Wells were aspired and washed again with 150 μl 0.1% Tween20/PBS (Sigma) washing solution three times for 7 min. plate was dried and scanned with Odyssey CLx imaging system at 700 nm and 800 nm (Licor). Wells signals were analyzed with Odyssey software. The signal was normalized by the CellTag700 signal.

In Vitro Activity Assays for MKK7 or MKK4 (Carried by Nanosyn):

Exemplary test compounds, according to some embodiments of the present invention, were diluted in DMSO to a final concentration that ranged from 10 μM to 0.0565 nM, while final concentration of DMSO in all assays was kept at 1%. Reference compound, Staurosporine, was tested in a similar manner. 1 nM MKK7 or MKK4 was preincubated with inactive JNK1 in a buffer comprising 100 mM HEPES, 5 mM MgCl₂, 1 mM DTT, 0.1% BSA, 0.01% Triton X-100 and 2 μM ATP for 2 hours. After incubation, activity of now activated JNK1 was tested in the presence of 30 μM ATP ATP for 17 h (MKK7) or 4 h (MKK4).

MKK7 Protein Purification:

MKK7 isoform 4 was obtained from the ORFeome, from which MKK7 isoform 1 (103-426 residues) with a C-terminal 6-His tag was cloned into pET28-TEVH vector and grown in E coli Resetta2 (DE3)pLYs cells. The cells were grown for 20 hours at 15° C. following induction with 0.2 mM IPTG.

The protein was purified using 5 ml TALON beads (Clonetech) equilibrated with PBS, 250 mM KCl, 5 mM DTT. Protein was eluted with the same buffer containing 500 mM imidazole. The eluted protein was injected immediately into a HiLoad Superdex 20016/60 prepgrade column equilibrated with 50 mM HEPES pH 6.7, 125 mM NaCl, 5 mM DTT. The peak containing MKK7 was concentrated and injected into a Superdex 75 HR 10/30 analytical column equilibrated with the same buffer.

Intact Protein Mass Spectrometry:

Purified MKK7 sample was incubated at concentration of 10 μM for 1 hour or 16 hours at 4° C. with 20 μM of either exemplary compounds GO4, GO32 or GO80. Samples were injected after indicated incubation times into LC/MS (Waters ACUITY UPLC class H), in positive ion mode using electrospray ionization. C4 column (300 Å, 1.7 μM, 21 mm×100 mm) was held at 40° C., and the autosampler at 10° C. Desolvation temperature was 500° C. with flow rate of 1000 liter/hour. The voltage used were 0.69 kV for the capillary and 46 V for the cone. Spectra were deconvoluted using MaxEnt1 software.

Structural Determination and Crystallography:

The stabilized mutants, C218S and C276S of MKK7 were prepared as previously reported [Kinoshita, T. et al., Biochem. Biophys. Res. Comm., 2017, 493(1), pp. 313-317]. Hexa-histidine-tagged MKK7 mutant was produced by transforming the E. coli strain, Rossetta2 (DE3) pLysS (MerckMillipore, Darmstadt, Germany), with the MKK7 gene inserted into pET22b (Merck Millipore). The protein was purified by TALON Cobalt-charged resin (Takara Bio, Otsu, Japan) and SPD Sepharose FF cation-exchange column (GE Healthcare, Little Chalfont, UK). The purified mutants of MKK7 were crystallized under the same conditions as the wild type, consisting of 22-25% (w/v) PEG3350, 0.2 M sodium citrate tribasic and 0.1M HEPES buffer, pH 7.5. The X-ray diffraction data sets were collected on a Pilatus3 S6M detector (Dectris) at the Photon Factory BL17A beamline or on a Quantum Q315s detector (ADSC) at the Aichi Synchrotron Radiation Center BL2S1 beamline, and integrated by XDS. The initial phase was determined by molecular replacement using the 5Y90 structure as a starting model. Structure refinement and model modification were iterated using Refmac5 and Coot in the CCP4 program suite. The data collection and refinement statistics are shown in Table 4 below. The final coordinates of the C218S/GO80 and C276S/GO80 complexes were deposited in the Protein Data Bank with IDs: 5Z1E and 5Z1D, respectively.

TABLE 4

| Mutant/Inhibitor: | C218S/GO80 | | C276S/GO80 | |
|---|---|---|---|---|
| Data collection | | | | |
| Space group | P2₁2₁2₁ | | P2₁2₁2₁ | |
| Unit cell (Å) | a = 53.91 | | a = 54.58 | |
| | b = 70.83 | | b = 68.83 | |
| | c = 92.71 | | c = 88.46 | |
| Observations | 113577 | | 93605 | |
| Unique reflections | 16329 | | 14659 | |
| Resolutions (Å) | 46.60-2.30 | (2.44-2.30) | 51.76-2.28 | (2.36-2.28) |
| Completeness (%) | 99.8 | (98.7) | 98.8 | (100) |
| R_{merge} (%)^{a} | 10.2 | (87.1) | 10.7 | (77.2) |
| I/σ | 9.7 | (1.2) | 10.2 | (2.2) |
| Refinement | 46.60-2.30 | (2.36-2.30) | 51.76-2.28 | (2.36-2.30) |
| statistics | | | | |
| Resolution (Å) | | | | |
| Reflections | 16329 | | 14635 | |
| Total atoms | 2504 | | 2333 | |
| R-factor (%) | 19.4 | (31.3) | 19.2 | (24.9) |
| R_{free} (%) | 28.4 | (30.2) | 25.8 | (30.3) |

| R.m.s. deviations | | |
|---|---|---|
| Bond length (Å) | 0.013 | 0.014 |
| Bond angle (°) | 1.6 | 1.7 |

Values in parentheses are for the highest-resolution shell. ^{a}R_{merge}=ΣₕΣⱼ|Iₕⱼ−<Iₕ>|/ΣₕΣⱼ|Iₕⱼ|, where h represents a unique reflection and j represents symmetry-equivalent indices. I is the observed intensity and <I> is the mean value of I.

Kinome Selectivity Screen:

Kinase selectivity was assessed using the Life Technologies SelectScreen Kinase Profiling Service in a custom panel of 76 kinases selected to provide broad representative coverage of all kinase families. Kinase activity assays employed either the Z′-LYTE or Adapta assay technologies with ATP concentrations at or near the KM, ATP for each kinase and a test compound concentration of 1 μM.

Activity Based Fluorescent Labeling for Chemical Probe Selectivity Analysis: The activity based fluorescent labeling, used for chemical probe selectivity analysis, were conducted as follows. HEK293 cells were lysed using Ripa buffer (Sigma) supplemented with protease inhibitor and phosphatase inhibitor cocktails (Sigma) and lysates were produced according to manufacturer's protocol.

E. coli strain Rosseta2 BL21 (DE3) over-expressing MKK7 (103-426 residues) were grown in 10 ml LB until O.D.₆₀₀=0.6-0.8 in 37° C., then protein expression was induced with 0.1 mM IPTG for 4 hours. Lysis buffer containing 20 mM Tris-HCl, pH=7, 50 mM NaCl, 5 mM DTT and 10% glycerol was added supplemented with protease inhibitors (Sigma), Benzonase nuclease (Sigma), followed by sonication on ice (30% Amplitude, 5 minutes, 10 seconds on, 40 seconds off), then centrifuged for 15000 rcf, 15 min 4° C.

50 μl lysates were pre-incubated with 0.5 μl, 5 mM GO32/GO80 in competition experiments for 1 hour 37° C., then incubated with 0.5 μl, 5 mM chemical probe containing alkyne groups for 1 hour at 37° C., then reacted with 5-TAMRA-Azide (5 μl, 0.5 mM), ascorbic acid (3 μl, 150 mM) and THPTA ligand (3 μl, 50 mM) in the presence of CuSO₄ (1 μl, 50 mM) in a final volume of 63 μl. Samples were supplemented with 4□ sample buffer and heated to 95° C. for 5 minutes. 50 μl of clicked lysates were run on 4-20% SDS-PAGE gel (Genscript) under reducing conditions, then imaged with Typhoon FLA 9500 laser scanner (GE).

### Example 4

### Results

Structure-Activity Relationship:

Based on the predicted docking results, a small analog series of GO4 was designed and synthesized. Moving to an indazole analog improved inhibition (GO32 IC₅₀=9 nM). Combined with an easier synthetic route via Suzuki-Miyaura coupling of readily available indazole building blocks we pursued this series further.

The docking model suggested that substitutions at the 6 or 7 position of the indazole may improve binding. Substitutions at the 7-position of the indazole were barely tolerated with a fluorine showing no change in IC₅₀, nitro- and chloro-substitutions showing slight decreases in activity, and a methyl substitution disrupted binding by two orders of magnitude. Many substitutions were however tolerated at the 6-position including nitro, methyl, methoxy, bromo, chloro and phenyl (see, Table 2).

An exemplary set of the most potent analogs, GO4, GO32 and GO80 were tested via intact protein mass spectrometry to show that these act through irreversible covalent labeling of the protein. After 1 hour or 16 hours incubation at 4° C. (molecule concentration 20 μM, protein concentration 10 μM) the compounds showed 65.16%, 78.14% and 59.58% covalent labeling respectively after 1 hour of incubation, and 95.15%, 96.29% and 92.87% covalent labeling respectively after 16 hours of incubation.

Potent Inhibition of JNK Phosphorylation in Cells:

To rapidly assess the cellular activity (EC₅₀) with full dose response curves of the MKK7 covalent inhibitors, according to some embodiments of the present invention, an in-cell western assay was adapted to a 384 well plate format over traditional western blot, and monitored phospho-JNK (pJNK) levels in response to osmotic shock. We first characterized the dynamics of pJNK in response to sorbitol. Sufficient dynamic range for pJNK is achieved at 20 minutes in HEK293 cells and at 40 minutes in 3T3 and Beas2B cells. The compound's activity was assessed by pre-treating cells with either compound or DMSO for 2 hours, followed by Sorbitol stimulation. The response of pJNK, and its substrates, pc-Jun and pATF2, was measured, as well as pP38, which is a substrate of MKK4 but not MKK7, and pERK in HEK293 cells, and the results are presented in Table 5 below.

TABLE 5

| Exemplary | pJNK inhibition | p-cJun inhibition | pATF2 inhibition |
|---|---|---|---|
| compound | EC₅₀ in μM | EC₅₀ in μM | EC₅₀ in μM |
| GO4 | 5.2 | | |
| GO32 | 2.06 | 2.26 | 2.79 |
| GO37 | | 2.52 | 3.71 |
| GO80 | 1.26 | 1.67 | 5.19 |

Despite similar in vitro activities, the exemplary MKK7 covalent inhibitor compounds, according to some embodiments of the present invention, showed various and significantly lower activity in cells. This lower activity may be due to competition with high levels of cellular ATP, potential depletion by cellular nucleophiles such as glutathione (GSH) and perhaps incomplete permeability. Since these inhibitors are time dependent another contribution to the apparent lower activity is the short incubation time with cells, 2 hour, compared to the relatively long incubation time required by the in vitro assay of 19 hours. Still, the lead compounds showed cellular EC₅₀ below 1 μM.

As can be seen in Table 5, the structure-optimized inhibitors GO32, and GO80 showed an EC₅₀ of 2.06 μM and 1.26 μM respectively for reduction of pJNK, compared to EC₅₀ of 5.2 μM of GO4. With similar values for pcJun (2.26 μM and 1.67 μM, respectively) and pATF2 (2.79 μM 5.19 μM, respectively). At 10 μM GO80 and GO37 did not affect pP38 or pERK levels while GO32 showed about 25% decrease in pERK with no effect on pP38.

To validate the covalent mechanism of inhibition, as well as the predicted binding pose, two negative control compounds, PCM520 and PCM521 (see chemical structure schemes below), were synthesized; the first with a reduced acrylamide, no longer able to form a covalent bond, and the latter methylated on the indazole nitrogen predicted to form a crucial hydrogen bond with the kinase hinge. Indeed, neither control compound showed any effect on pJNK up to 100 μM.

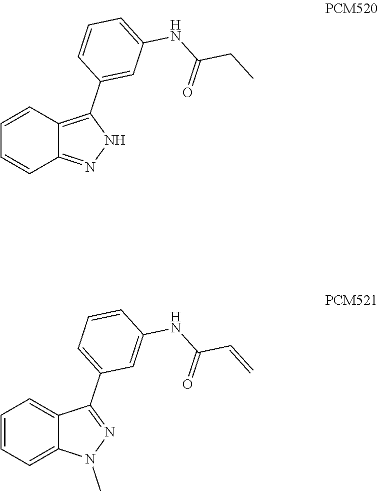 [SMILES: CCC(=O)Nc1cccc(-c2[nH]nc3ccccc23)c1 ; C=CC(=O)Nc1cccc(-c2nn(C)c3ccccc23)c1]

As an orthogonal assay for the ICW we used a KTR reporter cell line for JNK activation via live cell imaging. First, IC₅₀ of compounds GO4, GO32, GO37, GO80 was assessed in this cell line (Beas2B) via ICW and found similar though slightly higher values as those of the other cell lines. Thereafter, the kinetics of JNK activation was followed via live cell imaging of several single cells with and without the test and control compounds; the EC₅₀ results in HEK293 cells were 3.3 μM for GO80, and over 10 μM for both PCM520 and PCM521. Overall the inhibitors at low concentration demonstrated both a reduction of baseline JNK phosphorylation as well as a reduction in peak pJNK in response to sorbitol.

Genetic Validation of On-Target Activity:

Wildtype 3T3 cells, 3T3 MKK7 double knock-out (MKK7^{-/-}) cells and MKK4/7 double knockouts (MKK4/7^{-/-}) were used to validate on target activity of the MKK7 covalent inhibitors presented herein. While the 3T3 WT cells showed the expected increase in pJNK levels following sorbitol stimulation, both MKK7^{-/-} and MKK4/7^{-/-} cells showed reduced levels of pJNK. Moreover, 3T3 WT cells demonstrated concentration dependent inhibition after pre-incubation with the inhibitors, according to embodiments of the present invention, with the following IC₅₀ values: GO4=18.2 μM, GO32=5.15 μM, GO37=5.33 μM, GO80=4.05 μM. Treatment of either of the knockout cells with the inhibitors presented herein had no effect on their pJNK levels, which remained at the reduced base-line, suggesting that MKK7 is indeed mediating the compound's inhibition of JNK phosphorylation.

Inhibitors are Selective Across the Kinome:

To assess the selectivity of the inhibitors provided herewith across the kinome, a kinome panel of 76 kinases was assayed against 1 μM of GO32 and G80. Both GO32 and GO80 inhibitors displayed remarkable selectivity, with very few kinases inhibited by more than 75% at this concentration, such as Aurora Kinase B, LRRK2 and MKK4, as well as FLT3 for GO80 and JAK3 for GO32 (see, Table 6 below).

TABLE 6

| | GO32 | GO80 |
|---|---|---|
| | % Inhibition | % Inhibition |
| Kinome enzyme | [1 μM] | [1 μM] |
| ABL1 | 2 | 1 |
| ACVR1B (ALK4) | −8 | 5 |
| AKT1 (PKB alpha) | −5 | −4 |
| AMPK A1/B1/G1 | −5 | −5 |
| AURKA (Aurora A) | 4 | 4 |
| AURKB (Aurora B) | 79 | 77 |
| BTK | 11 | 15 |
| CDK1/cyclin B | 19 | 22 |
| CDK9/cyclin T1 | 3 | 5 |
| CHEK1 (CHK1) | 10 | 2 |
| CLK1 | 14 | 2 |
| CSNK1G2 (CK1 gamma 2) | 3 | −13 |
| CSNK2A1 (CK2 alpha 1) | 0 | −2 |
| DAPK3 (ZIPK) | −3 | −2 |
| DCAMKL2 (DCK2) | −2 | −1 |
| DYRK3 | 11 | 22 |
| EGFR (ErbB1) | −6 | −4 |
| EPHA2 | −16 | −13 |
| EPHB1 | −4 | −2 |
| ERBB2 (HER2) | 1 | −1 |
| FGFR1 | 2 | −2 |
| FLT1 (VEGFR1) | −1 | 2 |
| FLT3 | 55 | 79 |
| FLT4 (VEGFR3) | −2 | 0 |
| FRAP1 (mTOR) | −2 | −2 |
| GRK4 | −5 | −3 |
| GSK3B (GSK3 beta) | −10 | −8 |
| IGF1R | 6 | 7 |
| IKBKB (IKK beta) | −3 | −5 |
| INSR | −10 | −7 |
| IRAK4 | 15 | 12 |
| JAK3 | 89 | 64 |
| KDR (VEGFR2) | 4 | 6 |
| KIT | 17 | 10 |
| LCK | 5 | 5 |
| LRRK2 | 95 | 94 |
| LRRK2 FL | 91 | 86 |
| MAP2K1 (MEK1) | 4 | 4 |
| MAP2K4 (MEK4) | 79 | 81 |
| MAP3K8 (COT) | −7 | −12 |
| MAP3K9 (MLK1) | 37 | 8 |
| MAP4K4 (HGK) | 58 | 42 |
| MAP4K5 (KHS1) | 22 | −11 |
| MAPK1 (ERK2) | −3 | 3 |
| MAPK14 (p38 alpha) | 9 | 16 |
| MAPK8 (JNK1) | 15 | 16 |
| MAPKAPK2 | 2 | 1 |
| MARK1 (MARK) | 14 | 4 |
| MET (cMet) | 9 | 13 |
| NEK1 | 5 | 2 |
| NEK7 | 8 | 7 |
| NTRK1 (TRKA) | 22 | 44 |
| PAK4 | −11 | −16 |
| PDGFRB (PDGFR beta) | −3 | −13 |
| PDK1 Direct | 22 | 26 |
| PHKG2 | 1 | 7 |
| PIK3CA/PIK3R1 (p110 alpha/p85 alpha) | −6 | −19 |
| PIM1 | 1 | 5 |
| PIM2 | −2 | 3 |
| PKN1 (PRK1) | 4 | 1 |
| PLK1 | 1 | 3 |
| PRKACA (PKA) | −8 | −10 |
| PRKCB1 (PKC beta I) | 1 | 5 |
| PRKCD (PKC delta) | 9 | 7 |
| PRKCE (PKC epsilon) | 20 | 21 |
| PRKG2 (PKG2) | −2 | 0 |
| RET | 6 | 5 |
| ROCK1 | −3 | 3 |
| ROCK2 | −2 | −4 |
| RPS6KA3 (RSK2) | 2 | −1 |
| RPS6KB1 (p70S6K) | 5 | 0 |
| SRC | 10 | −4 |
| STK22D (TSSK1) | 0 | −1 |
| SYK | 19 | 17 |
| TAOK2 (TAO1) | 12 | 14 |
| TEK (Tie2) | 25 | 1 |

GO32 and GO80 were found to be weak inhibitors of MKK4, which does not contain the corresponding cysteine 218 of MKK7, exhibiting IC₅₀s of 4.15 μM and 7.81 μM respectively. While GO37 is completely specific for MKK7 with no detectable MKK4 inhibition up to 10 μM.

It is also noted that GO32, which is disclosed in the Chang study, is more potent (nanomolar levels) and more specific towards MKK7, than towards Aurora kinase (micromolar level) by at least two orders of magnitude. Hence, according to some embodiments of the present invention, the compounds presented herein are characterized by exhibiting an inhibitory effect towards MKK7 higher by two orders of magnitude compared to the inhibitory effect towards an Aurora kinase.

MKK7-GO80 Co-Crystal Structure Validates Docking Prediction;

In order to elucidate the compounds' binding mode and to evaluate the docking predictions, crystal structure of MKK7 in a complex with some of the inhibitor compounds presented herein was obtained; for example, the complex crystal structure of MKK7 with GO80 (PDB ID 5Z1D), as well as of the more stable Cys218Ser mutant in complex with GO80 (PDB ID 5Z1E).

The binding mode of GO80, as revealed in the crystal structure, closely recapitulates the docking prediction for GO4 with 1.23 Å root mean square deviation (RMSD; neglecting the additional methyl in GO80). As predicted, the two indazole nitrogens mediate two hydrogen bonds to the kinase hinge region, and the hydrophobic part of the indazole occupies the pocket formed by Leu266, Val196, Met212, Ala163, Val150 and Cys276 (mutated in this construct to serine). The acrylamide moiety clearly forms a covalent bond with Cys218 (1.9 Å C-S distance). The structure explains much of the structure activity relationship (SAR) of the analog series we have previously made, specifically there is no sufficient space for any modification other than fluorine at the 7-position of the indazole, while the 6-position is less constricted.

The crystal structure of the Cys218Ser mutant captured the reversible encounter complex of the compound, before formation of the covalent bond. The unreacted acrylamide Cβ atom is within striking distance of Ser218 Oγ (2.5 Å) that exemplifies that the formation of the covalent bond is driven by specific reversible recognition of the inhibitor.

Interestingly, the docking screen was performed against the DFG-in conformation of MKK7 (PDB ID 2DYL), and the covalent complex indeed crystalized in the same conformation. Surprisingly, the Cys218Ser mutant crystalized in the DFG-out conformation, suggesting that perhaps due to its small size GO80 can bind both conformations.

Metabolic Stability:

To demonstrate the utility of the compounds provided herewith to in vivo models, the in vitro metabolic stability of some exemplary compounds, according to embodiments of the present invention, was established in liver microsoms from several species, including mouse, rat, dog, monkey and human (see, Table 7 below). A reference compound, REF1250, was used in this study:

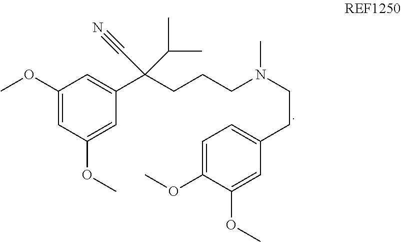 [SMILES: COc1cc(OC)cc(C(C#N)(CCCN(C)CCc2ccc(OC)c(OC)c2)C(C)C)c1]

TABLE 7

| | | | | Halflife | % remaining |
|---|---|---|---|---|---|
| | Compound | cLogP | Species | (min) | @ 40 min |
| Batch I | GO32 | 3.32 | Mouse | <5 | 0 |
| | | | Rat | <5 | 0 |
| | | | Dog | <5 | 0 |
| | | | Monkey | <5 | 0 |
| | | | Human | <5 | 0 |
| | GO80 | 3.84 | Mouse | <5 | 0 |
| | | | Rat | 6 | 1 |
| | | | Dog | <5 | 0 |
| | | | Monkey | <5 | 0 |
| | | | Human | <5 | 0 |
| | REF1250 | | Mouse | 7 | 2 |
| | | | Rat | 8 | 3 |
| | | | Dog | 12 | 12 |
| | | | Monkey | <5 | 0 |
| | | | Human | 9 | 5 |
| Batch II | GO4 | 3.52 | Mouse | <5 | 0 |
| | | | Rat | <5 | 0 |
| | | | Dog | <5 | 0 |
| | | | Monkey | <5 | 0 |
| | | | Human | <5 | 0 |
| | GO54 | 4.1 | Mouse | 11 | 18 |
| | | | Rat | 8 | 4 |
| | | | Dog | 6 | 1 |
| | | | Monkey | 5 | 1 |
| | | | Human | 16 | 20 |
| | GO55 | 1.91 | Mouse | 8 | 4 |
| | | | Rat | 29 | 40 |
| | | | Dog | 9 | 6 |
| | | | Monkey | <5 | 0 |
| | | | Human | 13 | 13 |
| | GO72 | 3.84 | Mouse | <5 | 0 |
| | | | Rat | <5 | 0 |
| | | | Dog | <5 | 0 |
| | | | Monkey | <5 | 0 |
| | | | Human | 12 | 11 |
| | GO73 | 3.18 | Mouse | <5 | 0 |
| | | | Rat | <5 | 0 |
| | | | Dog | <5 | 0 |
| | | | Monkey | <5 | 0 |
| | | | Human | <5 | 0 |
| | GO83 | 3.17 | Mouse | 5 | 0 |
| | | | Rat | 35 | 48 |
| | | | 0.00 | 0 | 0 |
| | | | Human | <5 | 1 |
| | | | Dog | <5 | 0 |
| | GO88 | 3.47 | Mouse | <5 | 0 |
| | | | Rat | 7 | 2 |
| | | | Dog | <5 | 0 |
| | | | Monkey | <5 | 0 |
| | | | Human | <5 | 0 |
| | GO89 | 3.93 | Mouse | <5 | 0 |
| | | | Rat | 6 | 1 |
| | | | 0.00 | 0 | 0 |
| | | | Monkey | <5 | 0 |
| | | | Human | 8 | 3 |
| | GO98 | 4.09 | Mouse | <5 | 0 |
| | | | Rat | 6 | 2 |
| | | | Dog | <5 | 0 |
| | | | Monkey | <5 | 0 |
| | | | Human | <5 | 0 |
| | GO106 | 3.93 | Mouse | <5 | 0 |
| | | | Rat | 16 | 17 |
| | | | Dog | <5 | 0 |
| | | | Monkey | <5 | 0 |
| | | | Human | <5 | 0 |
| | GO108 | 3.69 | Mouse | <5 | 0 |
| | | | Rat | 10 | 6 |
| | | | Dog | <5 | 0 |
| | | | Monkey | <5 | 0 |
| | | | Human | <5 | 0 |
| | REF1250 | | Mouse | 6 | 1 |
| | | | Rat | 6 | 1 |
| | | | Dog | 9 | 5 |
| | | | Monkey | <5 | 0 |
| | | | Human | 8 | 3 |
| Batch III | PCM475 | 2.7 | Mouse | <5 | 0 |
| | | | Rat | 18 | 22 |
| | | | Dog | 9 | 5 |
| | | | Monkey | 16 | 18 |
| | | | Human | <5 | 0 |
| | PCM476 | −0.55 | Mouse | >40 | 100 |
| | | | Rat | >40 | 61 |
| | | | Dog | >40 | 100 |
| | | | Monkey | >40 | 100 |
| | | | Human | >40 | 100 |
| | PCM487 | 2.4 | Mouse | 8 | 3 |
| | | | Rat | >40 | 72 |
| | | | Dog | >40 | 71 |
| | | | Monkey | 33 | 41 |
| | | | Human | 27 | 38 |
| | Assay Control | | Mouse | <5 | 0 |
| | | | Rat | <5 | 0 |
| | | | Dog | 8 | 3 |
| | | | Monkey | <5 | 0 |
| | | | Human | 6 | 1 |

As can be seen in Table 7, the compounds presented herein, having a relatively low c Log P exhibited notable stability. For instance, GO55 and GO83 with c Log P=1.91 and 3.17, respectively, exhibited half-lifes of 29 and 35 minutes in rat microsomes.

Structure Based Optimization Yields a New Vector:

Based on the complex crystal structure and the assays presented hereinabove, another series of analogs characterized by a low c Log P value has been synthesized (see, Table 3 hereinabove). The metabolic stability was determined for three of the compounds, namely PCM75, PCM76 and PCM87, and the stability of these less hydrophobic inhibitors increased significantly with more than 40 minutes half-life in dog and rat microsomes, particularly for PCM76 and PCM87 and significant stability in human microsomes of more than 40 minutes and 27 minutes respectively.

In terms of activity the piperidine inhibitors PCM531 and PCM532 proved to be inactive in ICW in 3T3 cells, as well as the carboxylic acid PCM76 which might be due to low permeability. It's amide counterpart PCM87, significantly improved activity (EC₅₀=4.06 μM). These results suggested that placing an alkyne functionality, as in PCM548, may be useful for the proteomic selectivity of the inhibitors provided herein.

Inhibitors are Selective Across the Proteome:

To establish target engagement of the alkyne functionality in PCM548, PCM548 was incubated in bacterial lysate overexpressing the catalytic domain of MKK7. After 2 hours incubation at room temperature, copper catalyzed cycloaddition with 5′-TAMRA-azide (“Click” chemistry) was used to fluorescently label the targets of PCM548, following by imaging on SDS-page gel. PCM548 clearly labels MKK7 in concentrations as low as 0.4 μM (results not shown). Moreover, both GO32 and GO80 are able to compete PCM548 binding when pre-incubated with the lysate (results not shown).

The proteomic selectivity of PCM548 was assessed in human cell lysate (MDA-MB-231). Despite the low endogenous expression of MKK7, a dose dependent band was observed, corresponding to the molecular weight of MKK7 that is detectable with up to 2 μM PCM548. While at 10 μM PCM548 labels many proteins, at 5 μM MKK7 seems to be the dominant target, and at 2 μM it seems to be the only target.

Compounds Block Activation of Primary Mouse B-Cells:

JNK is known to mediate activation of B-cells in response to lypopolysacharide (LPS) through the TLR4 signaling pathway as well as in response to B-cell receptor activation (stimulated e.g. with an anti-IgM antibody). Primary B-cells were isolated from the spleen of mice and assessed for their B-cell activation, via FACS analysis of CD86+ expression, in response to LPS in the presence or absence of the covalent MKK7 enzyme inhibitors presented herein, as well as a positive control JNK inhibitor, JNK-IN-8.

At 10 μM and 2 hours pre-incubation before stimulation, GO80 was able to inhibit 60% of the CD86+ response. PCM75, PCM87 and PCM548 were as potent as JNK-IN-8 and inhibited close to 90% of the response. It is noted herein that the non-covalent control only inhibited 26% of the response at the same concentration. A follow-up with full dose response for the three best inhibitors was conducted, out of which PCM87 and PCM548 displayed IC₅₀s of 4.9 μM and 5.3 μM respectively, similar to their IC₅₀ in inhibiting pJNK in response to sorbitol in 3T3 cells.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

1. A compound of general formula I′: 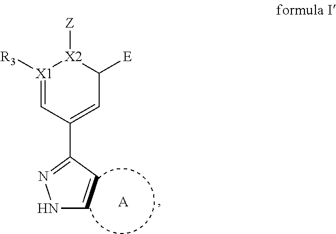
and any enantiomer, solvate, hydrate or a pharmaceutical acceptable salt thereof, wherein:
E is a reactive electrophile moiety 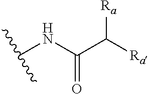
wherein Rₐ is selected from the group consisting of H, F, Cl, Br, Me, Et and Pr, and R_{a′} is selected from the group consisting of F, Cl and Br, or E is a reactive electrophile moiety selected from the group consisting of: 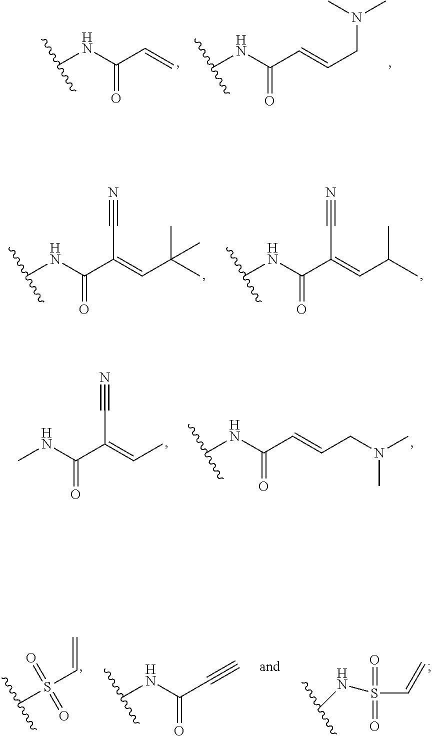
R₃ is H or a substituent selected from the group consisting of: 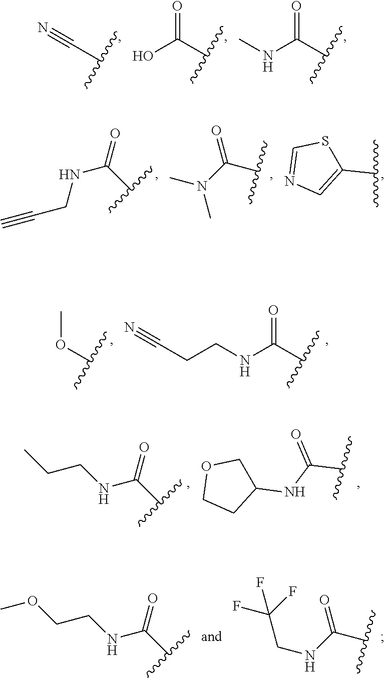
X₁ and X₂ are each independently C and N;
Z is CH₃ or H; and
ring A is a 5- or 6-membered, aromatic or aliphatic, substituted or unsubstituted ring having 0-2 heteroatoms therein, selected from the group consisting of: 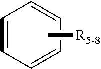
wherein each of R₅₋₈ is independently selected from the group consisting of H, F, Cl, Br, NO₂, Me, OMe and Ph, or ring A is selected from the group consisting of: 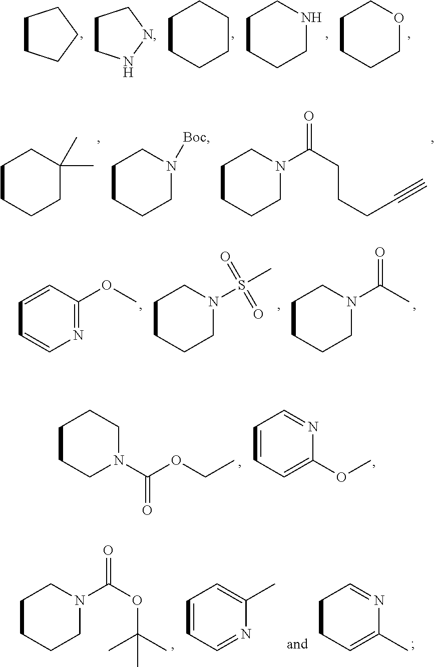
with the proviso that the compound is not 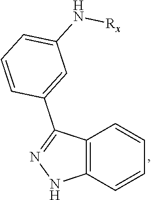
wherein:
Rₓ is

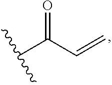 [SMILES: *C(=O)C=C]

2. The compound of claim 1, wherein said reactive electrophile moiety is capable of forming a covalent bond with a side-chain of residue that corresponds to a cysteine at position 218 of an MKK7 enzyme.

3. The compound of claim 1, capable of inhibiting human MKK7 enzyme by bonding covalently to CYS218 thereof.

4. The compound of claim 3, characterized by exhibiting inhibition of human MKK7 enzyme at a concentration lower by at least two orders of magnitude compared to an Aurora kinase enzyme.

5. The compound of claim 1, characterized by an octanol-water partition coefficient (Log P_{ow}) value that ranges from −1 to 6.

6. The compound of claim 1, selected from the group consisting of:

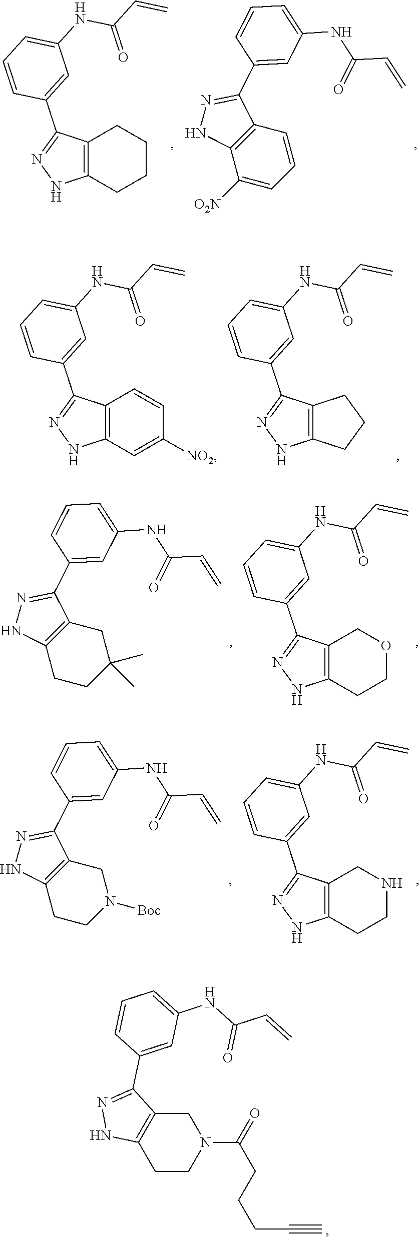

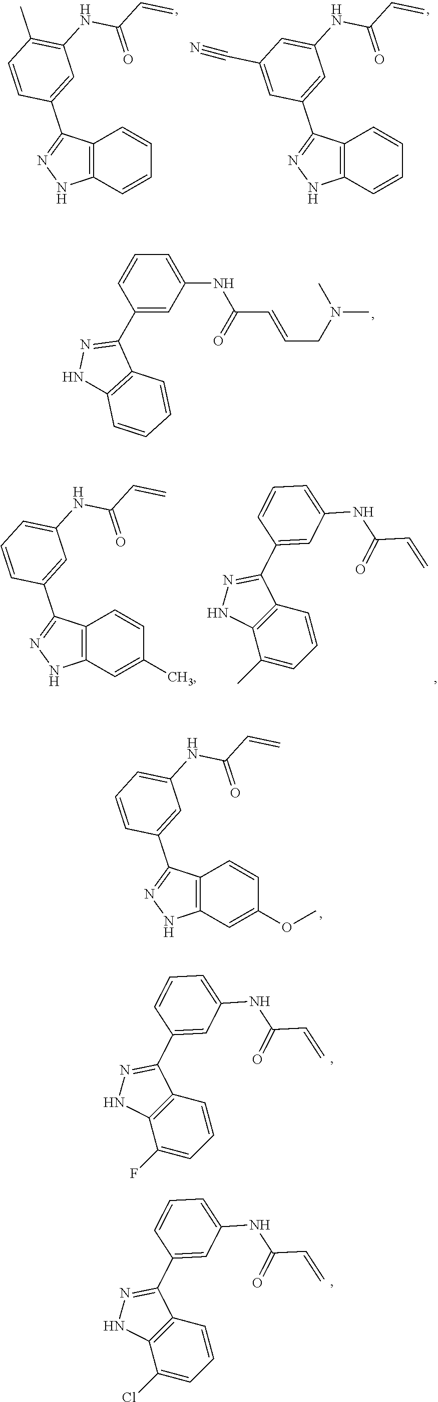

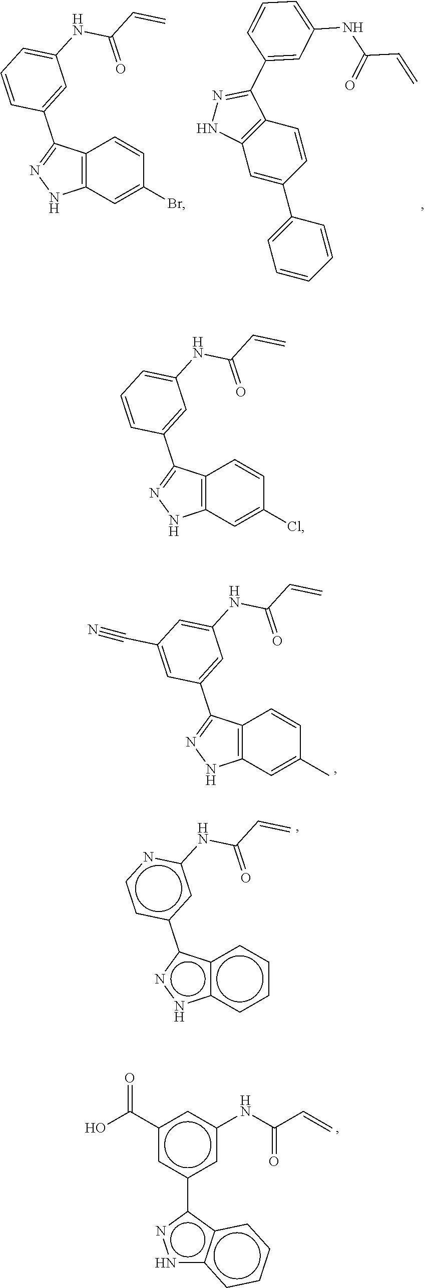

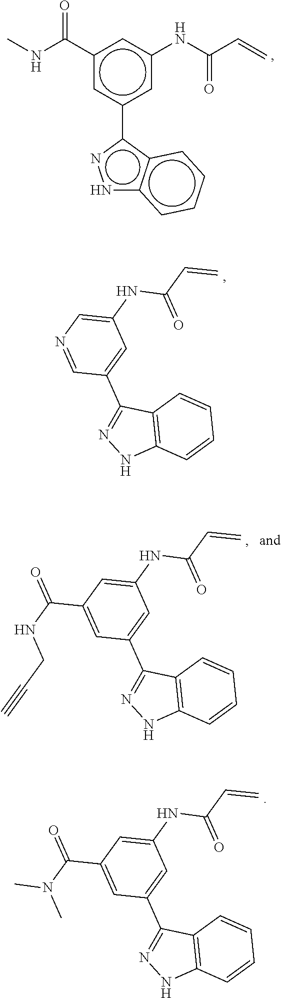

7. The compound of claim 6, selected from the group consisting of:

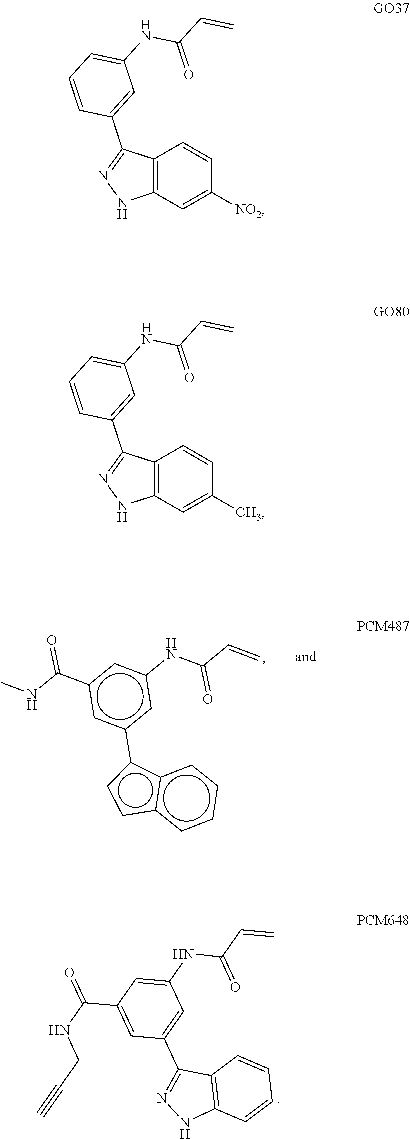

8-9. (canceled)

10. A method of treating a medical condition, disease or disorder associated with c-Jun-NH₂-terminal kinase (JNK) pathway regulation, comprising administering to a subject in need thereof a therapeutically effective amount of the compound of claim 1.

11. The method of claim 10, wherein said medical condition, disease or disorder is associated with MKK7 enzyme.

12-13. (canceled)

14. The method of claim 10, wherein said reactive electrophile moiety is capable of forming a covalent bond with a side-chain of residue that corresponds to a cysteine at position 218 of an MKK7 enzyme.

15. The method of claim 10, wherein said compound is capable of inhibiting human MKK7 enzyme by bonding covalently to CYS218 thereof.

16. The method of claim 10, wherein said compound is characterized by exhibiting inhibition of human MKK7 enzyme at a concentration lower by at least two orders of magnitude compared to an Aurora kinase enzyme.

17. The method of claim 10, wherein said medical condition, disease or disorder is selected from the group consisting of Parkinson's disease, Alzheimer's disease, Pick's disease, Crohn's disease, Behcet's disease, stroke, coronary artery disease, heart failure, abdominal aortic aneurysm, noonan syndrome, chronic hepatitis C virus infection, acute liver injury, non-alcoholic fatty liver disease, asthma, chronic obstructive pulmonary disease, amyotrophic lateral sclerosis, inflammatory bowel disease, polyglutamine disease, auditory hair cell degeneration, rheumatoid arthritis, systemic lupus eryththematosus, celiac disease, colorectal cancer, retinoblastoma, melanoma, breast carcinoma, ovarian cancer, obesity, insulin resistant, progressive supranuclear palsy, corticobasal degeneration, argyrophilic grain disease, familial frontotemporal dementia, and type 2 diabetes.

18. The method of claim 17, wherein said medical condition, disease or disorder is associated with MKK7 enzyme, and is not diabetes, cancer, or inflammation.

19. The method of claim 10, wherein said compound is characterized by an octanol-water partition coefficient (Log P_{ow}) value that ranges from −1 to 6.

20. The method of claim 10, wherein said compound is selected from the group consisting of:

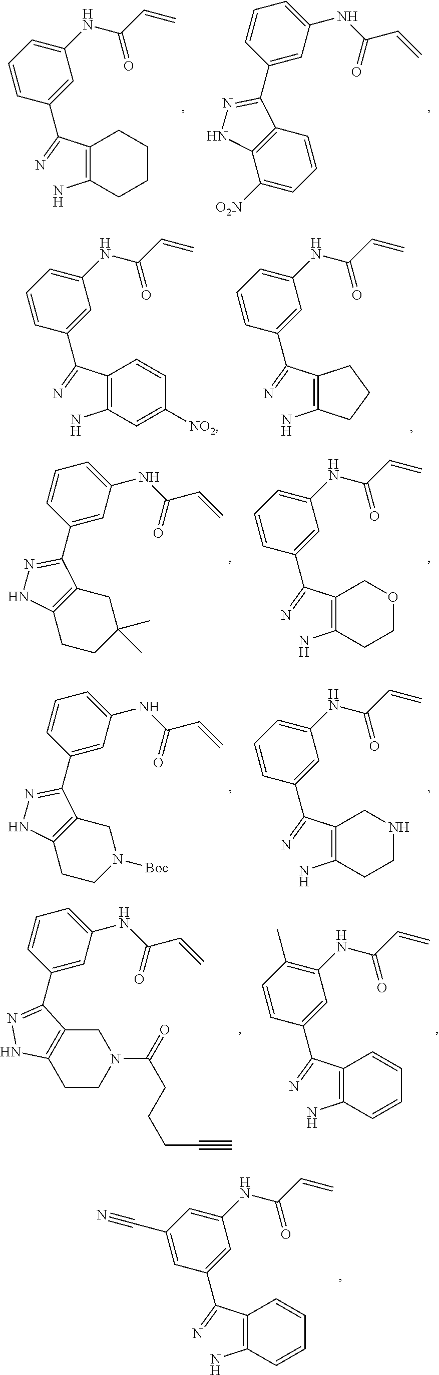

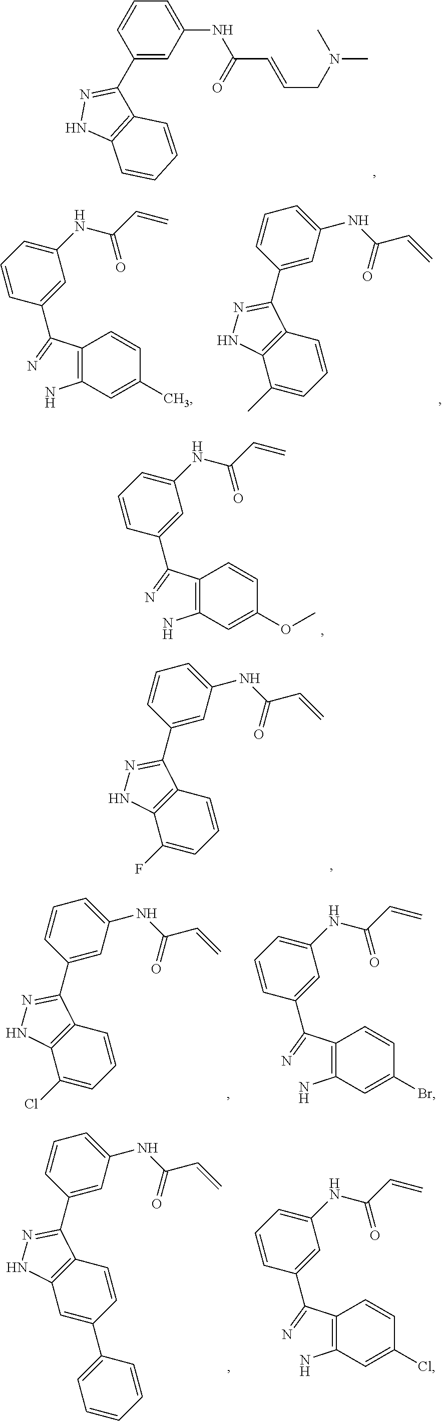

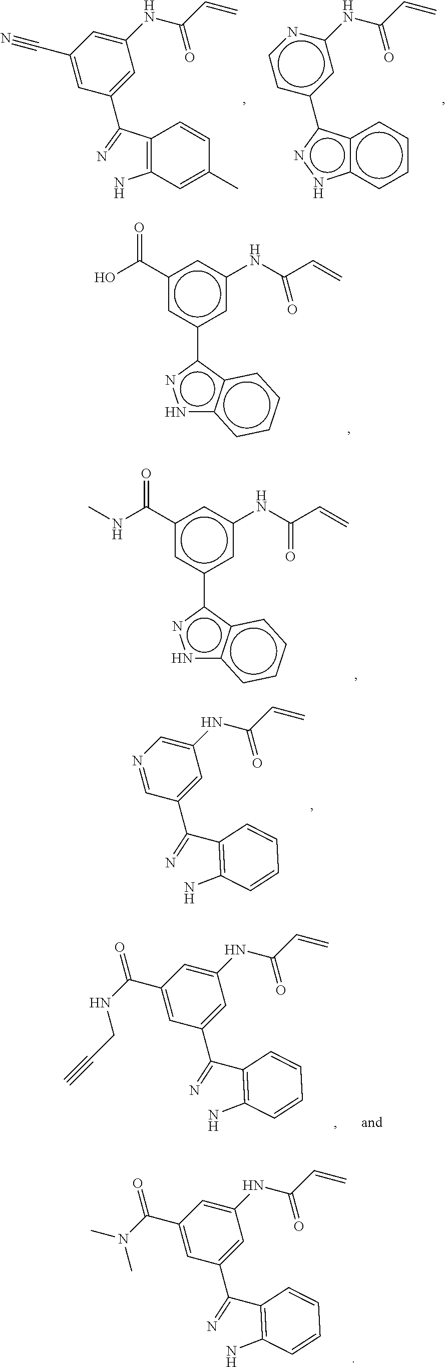

21. The method of claim 10, wherein said Z is H.

22. The method of claim 10, wherein each of said R₅₋₈ is independently selected from the group consisting of H, F, Cl, Br, Me, OMe and Ph.

23. The compound of claim 1, wherein said Z is H.

24. The compound of claim 1, wherein each of said R₅₋₈ is independently selected from the group consisting of H, F, Cl, Br, Me, OMe and Ph.
